(19) United States
(12) Patent Application Publication
(10) Pub. No.: US 2025/0230507 A1
(43) Pub. Date: Jul. 17, 2025

(54) METHODS AND SYSTEMS FOR CELL-FREE NUCLEIC ACID PROCESSING

(71) Applicant: Adela, Inc., Foster City, CA (US)

(72) Inventors: Daniel DINIZ DE CARVALHO, Toronto (CA); Justin BURGENER, Toronto (CA); Rajat SINGHANIA, Toronto (CA); Junjun ZHANG, Toronto (CA); Shu Yi SHEN, Toronto (CA); Iulia CIRLAN, Toronto (CA)

(21) Appl. No.: 18/954,991

(22) Filed: Nov. 21, 2024

Related U.S. Application Data
(63) Continuation of application No. PCT/US2023/023625, filed on May 25, 2023.
(60) Provisional application No. 63/345,803, filed on May 25, 2022.

(51) Int. Cl. C12Q 1/6886; C12N 15/10; C12Q 1/6806; C12Q 1/6809; C12Q 1/6844; C12Q 1/6869

(52) U.S. Cl. CPC C12Q 1/6886; C12N 15/1093; C12Q 1/6806; C12Q 1/6809; C12Q 1/6844; C12Q 1/6869; C12Q 2600/154

(57) ABSTRACT

Methods and systems for targeted detection of circulating tumor DNA (ctDNA) molecules are disclosed herein. A cell-free nucleic acid may be subjected to conditions to increase its methylation level, thereby yielding a hypermethylated cell-free nucleic acid, and subsequently identifying a sequence of the hypermethylated cell-free nucleic acid.

### CROSS-REFERENCE

This application is a continuation application of International Patent Application No. PCT/US2023/023625, filed May 25, 2023, which claims the benefit of U.S. Provisional Application No. 63/345,803, filed May 25, 2022, each of which is incorporated herein by reference in its entirety.

### BACKGROUND

Circulating tumor deoxyribonucleic acid (ctDNA) has increasingly demonstrated its potential as a non-invasive, tumor-specific biomarker for routine clinical use. ctDNA may be derived from tumor cells undergoing cell-death and released into circulation of various bodily fluids including blood. In certain cancer patients, the majority of blood-derived cell-free DNA may originate from healthy (e.g., non-cancerous) tissues. In addition, the fraction of ctDNA observed may range from <0.1% to 90% of the total cell-free DNA depending on several factors including the primary site of the tumor and disease burden. ctDNA provides non-invasive access to the tumor's molecular landscape and disease burden.

### SUMMARY

Recognized herein is the need for methods and systems for detecting circulating tumor deoxyribonucleic acid (ctDNA) with increased sensitivity, including for use with subjects with lower abundance of ctDNA.

One aspect of the present disclosure provides methods for generating a library of sequencing copies of methylated genomic regions, the methods comprising: a) providing a cell-free nucleic acid molecule of a subject and providing a sheared genomic nucleic acid molecule, wherein said sheared genomic nucleic acid molecule is separate from said cell-free nucleic acid molecule; b) subjecting said cell-free nucleic acid molecule or derivative thereof to conditions sufficient to increase a methylation level of said cell-free nucleic acid molecule or derivative thereof and said sheared genomic nucleic acid molecule or derivative thereof to conditions sufficient to increase a methylation level of said sheared genomic nucleic acid molecule or derivative thereof, thereby yielding a hypermethylated cell-free nucleic acid molecule and a hypermethylated genomic nucleic acid molecule, respectively; c) incubating said hypermethylated cell-free nucleic acid molecule and said hypermethylated genomic nucleic acid molecule to enrich for a first methylated genomic region of said hypermethylated cell-free nucleic acid molecule and a second methylated genomic region of said hypermethylated genomic nucleic acid molecule; and d) combining said first methylated genomic region of said hypermethylated cell-free nucleic acid molecule and said second methylated genomic region of said hypermethylated genomic nucleic acid molecule to generate said library of sequencing copies of methylated genomic regions. In some cases, said cell-free nucleic acid molecule is a cell-free deoxyribonucleic acid (DNA) molecule. In some cases, said cell-free DNA molecule is derived from a biological sample from said subject. In some cases, said biological sample comprises a blood sample or a plasma sample. In some cases, prior to (d), further comprising subjecting said first methylated genomic region of said hypermethylated cell-free nucleic acid molecule and said second methylated genomic region of said hypermethylated genomic nucleic acid molecule to conditions sufficient to amplify said first methylated genomic region of said hypermethylated cell-free nucleic acid molecule and said second methylated genomic region of said hypermethylated genomic nucleic acid molecule. In some cases, the methods further comprise sequencing said amplified first methylated genomic region of said hypermethylated cell-free nucleic acid molecule to generate a first plurality of sequencing reads and sequencing said amplified second methylated genomic region of said hypermethylated genomic nucleic acid molecule to generate a second plurality of sequencing reads. In some cases, said sequencing does not comprise bisulfite sequencing. In some cases, the methods further comprise obtaining a first input control of said cell-free nucleic acid molecule, wherein said input control comprises said hypermethylated cell-free nucleic acid molecule derived from b). In some cases, said first input control is not subjected to an enriching step as in c). In some cases, the methods further comprise subjecting said first input control to amplification and sequencing under sufficient conditions to generate a plurality of first input control sequencing reads. In some cases, the methods further comprise obtaining a second input control of said sheared genomic nucleic acid molecule, wherein said second input control comprises said hypermethylated sheared genomic nucleic acid molecule derived from b). In some cases, said second input control is not subjected to an enriching step as in c). In some cases, the methods further comprise subjecting said second input control to amplification and sequencing under sufficient conditions to generate a plurality of second input control sequencing reads. In some cases, sequencing reads of said first methylated genomic region of said hypermethylated cell-free nucleic acid molecule are derived by comparing said first plurality of sequencing reads to said plurality of first input control sequencing reads. In some cases, sequencing reads of said second methylated genomic region of said hypermethylated cell-free nucleic acid molecule is derived by comparing said second plurality of sequencing reads to said plurality of second input control sequencing reads. In some cases, the methods further comprise generating sequencing reads of enriched methylated DNA regions from a cell-free DNA sample from a subject who has or is suspected of having a disease or condition and generating sequencing reads of enriched methylated DNA regions from a cell-free DNA sample from a healthy subject. In some cases, the methods further comprise generating a first plurality of differentially methylated regions (DMRs) mapping to said library of sequencing copies of methylated genomic regions. In some cases, the methods further comprise generating a second plurality of differentially methylated regions (DMRs) mapping to a library of sequencing copies of a whole genome. In some cases, said first plurality of DMRs or said second plurality of DMRs comprise hypermethylation or hypomethylation. In some cases, said first plurality of DMRs is greater than said second plurality of DMRs. In some cases, when using said first plurality of DMRs to classify a diseased sample and a healthy sample, said diseased sample is better separated from said healthy sample as compared to using said second plurality of DMRs. In some cases, said separation of said diseased sample and said healthy sample is illustrated by a gene heatmap. In some cases, said separation of said diseased sample and said healthy sample is illustrated by a uniform manifold approximation and projection (UMAP) plot. In some cases, a runtime for classifying said diseased sample and said healthy sample is reduced as compared to using said second plurality of DMRs. In some cases, said disease or condition is a cancer or a tumor. In some cases, said cancer is breast cancer, bladder cancer, colorectal cancer, endometrial cancer, prostate cancer, renal cancer, pancreatic cancer, or lung cancer. In some cases, said cancer is a late-stage cancer. In some cases, said cancer is an early-stage cancer. In some cases, b) comprises using a binder that binds to one or more methylated nucleotides of a methylated region of said cell free nucleic acid molecule or said sheared genomic nucleic acid molecule. In some cases, said binder comprises a protein comprising a methyl-CpG-binding domain. In some cases, said protein is a MBD2 protein. In some cases, said binder comprises an antibody. In some cases, said antibody is a 5-mC antibody. In some cases, said antibody is a 5-hydroxymethyl cytosine antibody. In some cases, said binder exhibits a reduced level of a non-specific binding to non-methylated nucleotides of said cell free nucleic acid molecule or said sheared genomic nucleic acid molecule. In some cases, prior to c), the methods further comprise mixing said hypermethylated cell-free nucleic acid molecule or said hypermethylated genomic nucleic acid molecule with a filler deoxyribonucleic acid (DNA) molecule. In some cases, said filler DNA is used in step b) to enrich for a plurality of methylated regions in said cell-free nucleic acid molecule or derivative thereof or said sheared genomic nucleic acid molecule or derivative thereof. In some cases, said filler DNA has a length of about 50 base pairs (bp) to about 800 base pairs. In some cases, said length is about 100 bp to about 600 bp. In some cases, said length is about 200 bp to about 600 bp. In some cases, said sequencing comprises peak calling to identify enriched portions of said first plurality of sequencing reads or said second plurality of sequencing reads. In some cases, hypermethylated regions are identified using narrow peak calling. In some cases, hypermethylated regions are identified using broad peak calling.

Another aspect of the present disclosure provides methods for cell-free nucleic acid processing, comprising: a) providing a cell-free nucleic acid molecule of a subject; b) subjecting said cell-free nucleic acid molecule or derivative thereof to conditions sufficient to increase a methylation level of said cell-free nucleic acid molecule or derivative thereof, thereby yielding a hypermethylated cell-free nucleic acid molecule; and c) identifying a sequence of said hypermethylated cell-free nucleic acid molecule or derivative thereof.

In some cases, said cell-free nucleic acid molecule in (a) is obtained from a blood of said subject. In some cases, said blood comprises plasma, and wherein said cell-free nucleic acid molecule in (a) is obtained from said plasma.

In some cases, said identifying of (c) comprises sequencing said hypermethylated cell-free nucleic acid molecule. In some cases, detecting methylation patterns comprises identifying highly methylated regions. said sequencing comprises detecting methylation patterns.

In some cases, the methods further comprise immunoprecipitating said hypermethylated cell-free nucleic acid molecule before sequencing. In some cases, said sequencing comprises sequencing said immunoprecipitated hypermethylated cell-free nucleic acid molecule. In some cases, immunoprecipitating comprises using a methylated nucleic acid binder to bind said hypermethylated cell-free nucleic acid molecule. In some cases, said methylated nucleic acid binder comprises an antibody for methylated nucleic acid. In some cases, said antibody for methylated nucleic acid comprises an anti-5-Methylcytosine (5-mC) antibody. In some cases, said sequencing comprises subjecting said cell-free nucleic acid molecule or derivative thereof to a cfMeDIP-seq protocol.

In some cases, said cell-free nucleic acid molecule or derivative thereof is subjected to in vitro conditions sufficient to increase a methylation level of said cell-free nucleic acid molecule. In some cases, said conditions sufficient to increase a methylation level of said cell-free nucleic acid molecule comprises an enzyme. In some cases, the enzyme comprises CpG Methyltransferase. In some cases, said cell-free nucleic acid molecule in (a) is obtained from a body fluid comprising at least one of urine, amniotic fluid, cerebrospinal fluid, prostatic fluid, tear, or saliva.

In some cases, the methods further comprise: a) providing a sheared genomic nucleic acid molecule, wherein said sheared genomic nucleic acid molecule is separate from said cell-free nucleic acid molecule; b) subjecting said sheared genomic nucleic acid molecule or derivative thereof to conditions sufficient to increase a methylation level of said sheared genomic nucleic acid molecule or derivative thereof, thereby yielding a hypermethylated genomic nucleic acid molecule; and c) identifying a sequence of said hypermethylated genomic nucleic acid molecule or derivative thereof.

In some cases, the methods further comprise prior to (a), obtaining said sheared genomic nucleic acid molecule from said subject. In some cases, said sheared genomic nucleic acid sample is not from said subject. In some cases, said sheared genomic nucleic acid molecule comprises similar sequence length to that of cell-free nucleic acid. In some cases, said identifying in c) comprises sequencing said hypermethylated genomic nucleic acid molecule or derivative thereof.

In some cases, the methods further comprise using said sequencing to identify a methylation pattern of said hypermethylated genomic nucleic acid molecule or derivative thereof. In some cases, the methods further comprise using said sequencing to identify a highly methylated region of said hypermethylated genomic nucleic acid molecule, wherein said highly methylated region is a region that is at least 50% methylated.

In some cases, the methods further comprise processing said highly methylated regions identified in said hypermethylated cell-free nucleic acid with said highly methylated regions identified in said hypermethylated genomic nucleic acid and identifying a window of overlapping highly methylated regions. In some cases, said window of overlapping highly methylated regions contains overlapping highly methylated sequences that are about 100 base pairs (bp) to 500 bp in length. In some cases, said window of overlapping highly methylated regions contains overlapping highly methylated sequences that are about 250 bp to 350 bp in length. In some cases, a plurality of identified windows of overlapping highly methylated regions collectively comprises from about 10% to about 20% of a whole genome. In some cases, said window comprises from about 0.5 million to 2.5 million highly methylated regions.

Another aspect of the present disclosure provides methods of identifying differentially methylated regions (DMRs), comprising analyzing and comparing methylation patterns of a plurality of windows of overlapping sequence reads between a diseased sample and a control sample, and identifying hyper-or hypomethylated regions in said diseased sample as differentially methylated regions (DMRs), as compared to said control sample.

In some cases, analyzing methylation patterns of said plurality of windows of overlapping sequence reads comprises immunoprecipitating cell-free nucleic acid in said diseased sample and then analyzing methylation patterns of said immunoprecipitated cell-free nucleic acid. In some cases, said method identifies DMRs that are not found using a whole-genome as a background. In some cases, said method yields a larger number of DMRs as compared to using a whole-genome as a background. In some cases, said method yields at least twice said number of DMRs as compared to using a whole-genome as a background. In some cases, said method can identify DMRs more quickly than using a whole-genome as a background.

Another aspect of the present disclosure provides methods of analyzing a sample, comprising analyzing said differentially methylated regions (DMRs) identified in said diseased sample, wherein said method identifies said sample as a diseased sample or a normal sample

In some cases, said method provides better distinction compared to using genome-wide DMRs. In some cases, said method provides a distinction between a cancerous sample and a normal sample. In some cases, said method provides a distinction between different types of cancer.

In some cases, the methods further comprise providing a therapeutic intervention to said subject upon identifying said sequence. In some cases, providing said therapeutic intervention comprises providing a therapy to said subject. In some cases, providing said therapeutic intervention comprises providing a report that identifies said sequence of said hypermethylated cell-free nucleic acid molecule or derivative thereof. In some cases, providing said therapeutic intervention comprises providing a report that provides a therapeutic recommendation to said subject for treating a disease associated with said hypermethylated cell-free nucleic acid molecule or derivative thereof. In some cases, providing said therapeutic intervention comprises treating said subject for a disease associated with said hypermethylated cell-free nucleic acid molecule or derivative thereof. In some cases, said disease is cancer.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### Incorporation by Reference

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows a diagram illustrating a process for collecting flow-through of unmethylated/hypomethylated deoxyribonucleic acid (DNA) fragments.

FIG. 2 shows a plot of the results of cfMeDIP-seq analysis in cancerous versus healthy tissue. The volcano plot shows the fold-change of the normalized read counts on the x-axis and the negative log10 of the q-values on the y-axis, using all the 300-bp windows in the “novel background,” also known as the background region. DMRs are in gray.

FIG. 3 shows a plot of the results of cfMeDIP-seq analysis in cancerous versus healthy tissue. The volcano plot shows the fold-change of the normalized read counts on the x-axis and the negative log10 of the q-values on the y-axis, using all the 300-bp genome-wide windows. DMRs are in gray.

FIG. 4 shows the ability of the identified DMRs to separate the cancer samples from the control samples. The heatmap shows Z-scores of the RPKMs obtained from the MEDIPS software. DMRs from the background region are compared with the controls, shown in blue in the first annotation row above the heatmap. The controls are clustered distinctly from the various cancer samples. The standard Ward with Euclidean distance algorithm was used.

FIG. 5 shows the ability of the identified DMRs to separate the cancer samples from the control samples. The heatmap shows Z-scores of the RPKMs obtained from the MEDIPS software. Genome-wide DMRs are compared with the controls, shown in blue in the first annotation row above the heatmap. Here, some cancer cases cluster together with the healthy controls. The standard Ward with Euclidean distance algorithm was used.

FIG. 6 shows the heightened ability of the DMRs from the background region to distinguish between cancer and control samples in a plot generated by UMAP, a dimensionality reduction software.

FIG. 7 shows the genome wide DMRs' lessened ability to distinguish between cancer and control samples in a plot generated by UMAP, a dimensionality reduction software.

FIG. 8 shows that the run time to get the DMRs in the background region is almost an order of magnitude lower than the run time to get the genome-wide DMRs.

FIG. 9 shows a Venn diagram in which the Hyper DMRs generated from the genome-wide background are almost all present in the list of those that are generated with the background region, with the latter finding many more DMRs of interest that are not found with the genome-wide analysis.

FIG. 10 shows a computer system that is programmed or otherwise configured to implement methods provided herein.

FIG. 11 shows de novo peak calls for fully methylated samples and consensus peak annotation (mu merge) at a region in human chromosome 4.

FIG. 12 shows de novo peak calls for fully methylated samples and consensus peak annotation (mu merge) at a region in human chromosome 17.

FIG. 13 shows a histogram of counts over feature region of interest (ROI), where the feature regions are determined by the fully methylated samples.

FIG. 14 depicts an embodiment of workflow.

### DETAILED DESCRIPTION

The present disclosure provides methods and systems for the processing and analysis of nucleic acids present in biological samples through the generation of libraries of methylated genomic regions, which can be useful in determining a risk or likelihood of a subject having cancer or a tumor with high sensitivity and/or high specificity. Methods and systems provided herein can comprise the creation of hypermethylated cell-free nucleic acid molecules and hypermethylated sheared genomic nucleic acid molecules, which can be processed to differentiate between, for example, cancerous and non-cancerous tissue in circulating free DNA (cfDNA).

For example, the use and analysis of hypermethylated and/or differentially methylated nucleic acids across a specified range rather than the entire genome can allow for highly sensitive and highly specific detection and/or characterization of circulating tumor DNA (ctDNA) in a fluid sample (e.g., a blood sample) obtained from a subject. In some cases, the use and analysis of hypermethylated nucleic acids and/or differentially methylated nucleic acids can allow for increased sensitivity, specificity, and/or efficiency in the determination of a subject's risk of having or having a risk of developing a tumor or cancer.

### Definitions

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “subject,” as used herein, generally refers to any member of the animal kingdom. The subject may be a human. The subject may be an individual exhibiting a disease (e.g., cancer) or an individual not exhibiting the disease. The subject may be considered to have a risk of developing the disease, such as cancer. The subject may be symptomatic or asymptomatic for a disease. The subject may be a patient. The subject may be a patient receiving medical care for a disease or condition (e.g., cancer).

The term “genome,” as used herein, generally refers to genomic information from a subject, which may be, for example, at least a portion or an entirety of a subject's hereditary information. A genome can be encoded either in DNA or in RNA. A genome can comprise coding regions (e.g., that code for proteins) as well as non-coding regions. A genome can include the sequence of all chromosomes together in an organism. For example, the human genome ordinarily has a total of 46 chromosomes. The sequence of all of these together may constitute a human genome.

The term “methylome,” used herein, generally refers to measure of an amount of DNA methylation and/or DNA methylation level at a plurality of sites or loci in a genome. DNA methylation is a process by which methyl groups are added to a DNA molecule. DNA methylation can act to modulate (e.g., repress) gene transcription. The methylome may correspond to all of the genome (whole genome methylation), a substantial part of the genome, or relatively small portion(s) of the genome. The term “methylome” as used herein can also refer to the set of methylation modifications (e.g., on a nucleic acid) in an organism, in a cell, or in a sample. A methylome can depend on the method of methylation measurement. For example, when using the antibody 5mC, a methylome can represent all the information of DNA methylation on the cytosines of a genome.

The term “nucleic acid,” used herein, generally refers to a polynucleotide comprising two or more nucleotides, i.e., a polymeric form of nucleotides of various lengths (e.g., at least 10, 20, 30, 40, 50, 100, 200, 300, 400, 500, 1000, 10000, or more nucleotides in length), either deoxyribonucleotides (dNTPs) or ribonucleotides (rNTPs), or analogs thereof. Non-limiting examples of nucleic acids include deoxyribonucleic (DNA), ribonucleic acid (RNA), coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or after assembly of the nucleic acid. The sequence of nucleotides of a nucleic acid may be interrupted by non-nucleotide components. A nucleic acid may be further modified after polymerization, such as by conjugation or binding with a reporter agent. A “variant” nucleic acid is a polynucleotide having a nucleotide sequence identical to that of its original nucleic acid except having at least one nucleotide modified, for example, deleted, inserted, or replaced, respectively. The variant may have a nucleotide sequence at least about 80%, 90%, 95%, or 99%, identity to the nucleotide sequence of the original nucleic acid.

Cell-free methylated DNA generally is DNA that can be one or more nucleic acid molecules circulating freely in the blood stream. In some cases, cell-free methylated DNA can be methylated at various regions of the DNA. Samples, for example, plasma samples may be taken to analyze cell-free methylated DNA. Studies reveal that much of the circulating nucleic acids in blood arise from necrotic or apoptotic cells and greatly elevated levels of nucleic acids from apoptosis is observed in diseases such as cancer. Particularly for cancer, where the circulating DNA bears hallmark signs of the disease including mutations in oncogenes, microsatellite alterations, and, for certain cancers, viral genomic sequences, DNA or RNA in plasma has become increasingly studied as a potential biomarker for disease. For example, a quantitative assay for low levels of circulating tumor DNA in total circulating DNA may serve as a better marker for detecting the relapse of colorectal cancer compared with carcinoembryonic antigen, the standard biomarker used clinically. Cell-free DNA (e.g., circulating cfDNA) may comprise circulating tumor DNA (ctDNA).

As used herein, “sequencing,” also known as “genomic sequencing,” is a process for determining the order of the chemical building blocks (e.g., adenine, cytosine, guanine, thymine, uracil) that make up a nucleic acid molecule (e.g., DNA, RNA, cDNA).

As used herein, “library preparation” generally includes list end-repair, A-tailing, adapter ligation, or any other preparation performed on the cell free DNA to permit subsequent sequencing of DNA. Library preparation can allow for a nucleic acid sample (e.g., DNA, cDNA) to adhere to the sequencing apparatus (e.g., a flow cell, a bead). Non-limiting examples of library preparation include ligation-based library preparation and tagmentation-based library preparation. Library preparation can result in the creation of a sequencing library, a pool of nucleic acid (e.g., DNA) fragments with adapters attached. The type of adapter attached during library preparation can depend on the sequencing platform/apparatus used.

The output of sequencing can be a “sequencing read.” As used herein, a “sequencing read” is an inferred sequence of base pairs or base pair probabilities corresponding to all or part of a nucleic acid fragment (e.g., a DNA fragment). The length of a sequencing read can depend on the sequencing platform/apparatus used. The length of a sequencing read can be about 10, about 20, about 30, about 40, about 50, about 60, about 70, about 80, about 90, about 100, about 150, about 200, about 250, about 300, about 350, about 400, about 450, about 500, about 550, about 600, about 650, about 700, about 750, about 800, about 850, about 900, about 950, about 1000, about 1100, about 1200, about 1300, about 1400, about 1500, about 1600, about 1700, about 1800, about 1900, about 2000, or more base pairs in length.

As used herein, “sequencing depth” refers to the ratio of the total number of bases obtained by sequencing to the size of the genome. Sequencing depth can also refer to the average number of times each base is measured in a genome during sequencing. Factors that can determine sequencing depth can include the error rate of the sequencing methods, the assembly algorithm used during sequencing, the repeat complexity of the nucleic acid molecule, region, or genome that is being sequenced, and the length of the sequencing read.

As used herein, “supplemental processed DNA” (e.g., “filler DNA”) generally may be noncoding DNA or it may consist of amplicons.

In some embodiments, the fragment length metric is fragment length. In some embodiments, the subject cell-free methylated DNA is limited to fragments having a length of <170 bp, <165 bp, <160 bp, <155 bp, <150 bp, <145 bp, <140 bp, <135 bp, <130 bp, <125 bp, <120 bp, <115 bp, <110 bp, <105 bp, or <100 bp. In other embodiments, the subject cell-free methylated DNA is limited to fragments having a length of between about 100—about 150 bp, 110-140 bp, or 120-130 bp.

In some embodiments, the fragment length metric is the fragment length distribution of the subject cell-free methylated DNA. In some embodiments, the subject cell-free methylated DNA is limited to fragments within the bottom 50th, 45th, 40th, 35th, 30th, 25th, 20th, 15th, or 10th percentile based on length.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

### Methods for Comparative DNA Methylation

Cell-free nucleic acids, such as cell-free DNA (cfDNA), which can be present in biological samples that can be collected non-invasively (e.g., blood, urine, saliva, cerebrospinal fluid (CSF), etc.), can be a heterogeneous population comprising both cfDNA derived from healthy tissues and cfDNA derived from tumor or cancer cells (e.g., ctDNA). Cancer development can be associated with focal gain of 5′ methylcytosines (5mC), for instance, at cytosine-phosphate-guanine (CpG) islands and CpG island shores. Cancer development can also be associated with global (e.g., genome-wide) cytosine demethylation (e.g., global loss of 5mC). In some cases, ctDNA can be distinguished from cfDNA molecules derived from healthy tissue (e.g., non-tumor and/or non-cancer tissue) by the methylation level (e.g., the percentage of nucleotide residues that are methylated) of the nucleic acid molecules. In some cases, nucleic acid molecules of or derived from tumor tissue and/or cancer tissue can be hypomethylated (e.g., can comprise a lower level of methylation, for instance, wherein there are fewer methylated nucleotide residues and/or a lower percentage of methylated nucleotide residues) compared to nucleic acid molecules of or derived from healthy tissue (e.g., nucleic acid molecules of or derived from healthy tissue that consist of or comprise nucleotide sequences corresponding to the same region(s) of the genome of the subject). For example, tumor-derived nucleic acid molecules (e.g., ctDNA molecules) can comprise one or more regions having fewer methylated nucleotide residues than nucleic acid molecules (e.g., cfDNA molecules) derived from healthy tissues (e.g., non-tumor and/or non-cancer tissues) in the same biological sample. In some cases, nucleic acid molecules of or derived from tumor tissue and/or cancer tissue can be hypermethylated (e.g., can comprise a higher level of methylation, for instance, wherein there are greater methylated nucleotide residues and/or a greater percentage of methylated nucleotide residues) compared to nucleic acid molecules of or derived from healthy tissue (e.g., nucleic acid molecules of or derived from healthy tissue that consist of or comprise nucleotide sequences corresponding to the same region(s) of the genome of the subject). For example, tumor-derived nucleic acid molecules (e.g., ctDNA molecules) can comprise one or more regions having greater methylated nucleotide residues than nucleic acid molecules (e.g., cfDNA molecules) derived from healthy tissues (e.g., non-tumor and/or non-cancer tissues) in the same biological sample. In some cases, all or a portion of a tumor-derived fraction of a plurality of cell-free DNA molecules (e.g., ctDNA) can be distinguished from cfDNA molecules derived from healthy tissue by one or more biophysical properties (e.g., the length of the cfDNA molecules or the presence of stereotypical 5′ and 3′ end sequence motifs) and/or one or more fragmentomics patterns. For instance, ctDNA molecules can have shorter nucleic acid lengths than cfDNA molecules derived from healthy tissues. In some cases, ctDNA molecules may comprise stereotypical 5′ and 3′ end motifs. In some cases, one or more of these distinguishing features may be used to deplete a population of nucleic acid molecules of cfDNA derived from healthy tissue and/or to enrich a population of nucleic acid molecules for ctDNA. In some cases, ctDNA can have shorter fragment length compared to cfDNA derived from a healthy tissue.

Nucleic acid molecules derived from tumor or cancer cells or tissue (e.g., ctDNA) may be present in a biological sample (and/or a population of nucleic acids derived from the biological sample) in substantially lower quantities than nucleic acid molecules (e.g., cfDNA) derived from healthy tissue. It can be difficult to detect or sequence (e.g., determine a sequence identity of) ctDNA present in a plurality of nucleic acid molecules (e.g., cfDNA) in or derived from a biological sample, for instance, because they are present in the sample in lower quantities relative to cfDNA derived from healthy tissue (e.g., which may require using a greater amount of potentially scarce biological sample and/or which may require significantly higher sequencing depth, if it is possible at all).

Depletion (e.g., removal) of all or a portion of the population of methylated DNA molecules (e.g., molecules having increased nucleotide methylation levels throughout or in a subset of the regions of the genome represented by the plurality of nucleic acid molecules of a biological sample) from a plurality of nucleic acid molecules (e.g., a plurality of cell-free nucleic acid molecules, or amplicons thereof, comprising a biological sample) may yield a remainder population of the plurality of nucleic acids of the biological sample that may be useful for determining a presence and/or sequence identity of ctDNA molecules in the biological sample. Depletion and/or removal may be performed by using a binder specific for methylated DNA molecules to pull them down. The pull-down is typically collected and the flow-through containing the unmethylated and/or hypomethylated DNA molecules is discarded. The current disclosure provides for methods and systems to collect such flow-through containing unmethylated and/or hypomethylated DNA molecules and to generate sequencing library using methylated and/or hypomethylated DNA molecules, or derivatives thereof.

In some cases, global or whole-genome methylation techniques can provide information about methylation across the genome, which can differentiate between healthy and cancerous tissue. In some cases, a plurality of nucleic acids (e.g., cfDNA molecules or amplicons thereof derived from a biological sample) may be subjected to genome-wide depletion of nucleic acid molecules methylated in one or more specific regions of the genomic sequence of the nucleic acid molecules (e.g., CpG islands, CpG island shores, or repetitive sequences of the genome, such as long interspersed nuclear elements (LINEs), short interspersed nuclear elements (SINEs), or LTRs (long terminal repeats)) to achieve increased sensitivity and/or increased specificity in assays for determining the presence or absence or the sequence identity of ctDNA molecules in the plurality. In some cases, a whole genome comprises all genomic regions. Alternatively, a whole genome can comprise all hypomethylated and/or all hypermethylated genomic regions. In some cases, a whole genome file comprises all the genomic regions of all the chromosomes of a sample (e.g., all human chromosomes). Alternatively, a whole genome file can comprise all the genomic regions of the autosomes (e.g., human chromosomes 1-22).

Alternatively, a subset of the global or whole-genome can be used to provide specific information about methylation at specified sites. Specified sites can include a set of background genomic regions that, when methylated, can provide additional information for distinguishing differentially methylated regions for the purposes of distinguishing cancer from control samples. A set of background genomic regions can be experimentally validated.

For example, a subset of whole-genome DNA can be obtained by using sheared DNA. Sheared DNA, also referred to as “cfDNA mimic” can comprise a subset of whole-genome DNA. In some cases, sheared DNA comprises randomly cleaved DNA. Alternatively, in some cases, sheared DNA comprises DNA cleaved at specified locations along the genome. In some cases, sheared DNA comprises DNA that is fragmented to a desired fragment range. Physical shearing can be performed using, for example, probe sonication or nebulization. Sheared genomic nucleic acid molecules (e.g., sheared DNA) can be about 20, about 30, about 40, about 50, about 60, about 70, about 80, about 90, about 100, about 150, about 200, about 250, about 300, about 350, about 400, about 450, about 500, about 550, about 600, about 650, about 700, about 750, about 800, about 850, about 900, about 950, about 1000, or more base pairs in length.

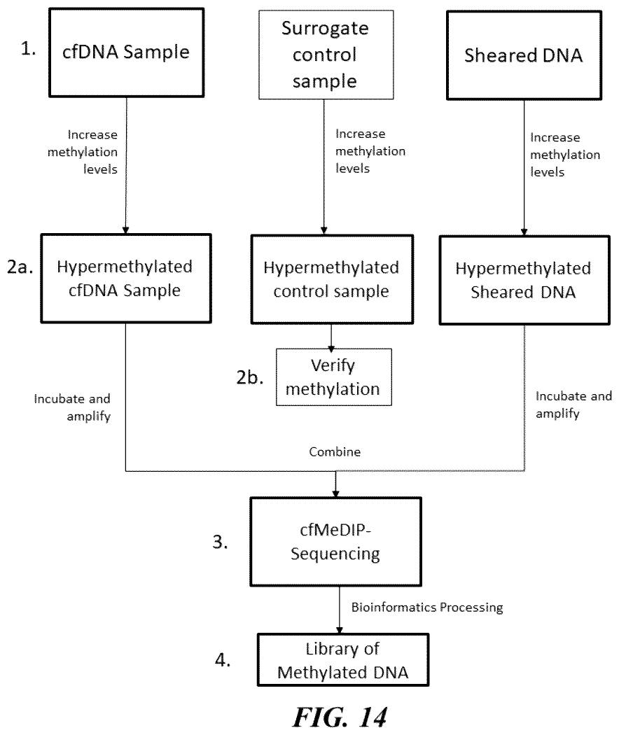

As shown in FIG. 14, nucleic acids (e.g., DNA, cell-free DNA (cfDNA)) obtained from a subject can be further treated to increase methylation level. These nucleic acids samples may be described as native cfDNA samples distinguished from cfDNA mimics. Native cfDNA samples may have a closer representation of the DNA fragmentation patters observed in clinical samples. The native cfDNA samples can be obtained as biobanked plasma samples corresponding to cancer patients and/or normal or healthy control subjects. In some cases, nucleic acids (e.g., DNA, cfDNA) can be partially methylated. In some cases, nucleic acids (e.g., DNA, cfDNA) can be fully methylated. In some cases, increasing methylation level is performed using in vitro methylation by an enzyme directed to alter methylation levels of nucleic acid fragments. In some embodiments, the enzyme is CpG methyltransferase. In some cases, in vitro methylation can result in partial methylation. In some cases, in vitro methylation can result in full methylation. In some cases, in vitro methylation can result in hypermethylation. In some cases, a methylated DNA binder (e.g., a 5mC antibody) is used to obtain or enrich for hypermethylated nucleic acids. Hypermethylated nucleic acids can be further incubated and amplified.

Also shown in FIG. 14, sheared genomic DNA fragments are treated to increase methylation levels. In some cases, sheared genomic DNA fragments can be partially methylated. In some cases, sheared DNA can be fully methylated. In some cases, increasing methylation level of the sheared DNA is performed using in vitro methylation by an enzyme directed to alter methylation levels of nucleic acid fragments. In some embodiments, the enzyme is CpG methyltransferase. In some cases, in vitro methylation can result in partial methylation. In some cases, in vitro methylation can result in full methylation. In some cases, in vitro methylation can result in hypermethylation. In some cases, a methylated DNA binder (e.g., a 5mC antibody) is used to obtain or enrich for hypermethylated sheared nucleic acids. Hypermethylated sheared nucleic acids can be further incubated and amplified.

In some cases, the processing of a nucleic acid sample occurs prior to the processing of a sheared DNA sample. Alternatively, the processing of a nucleic acid sample can occur simultaneously with the processing of a sheared DNA sample. Alternatively, the processing of a nucleic acid sample can occur after the processing of a sheared DNA sample.

Additionally, a surrogate control sample can be used to verify whether an enzyme for increasing methylation level of DNA samples actually works. A surrogate control sample can be processed in the same way as a native cfDNA sample as described above. A surrogate control sample can be used to ensure that a typically methylated region can be pulled down during the enrichment and processing steps.

In some cases, native cfDNA samples are split into two native cfDNA treatment groups prior to MeDIP enrichment. The first native cfDNA treatment group can be enriched as described above. The second native cfDNA treatment group can be used as a first input control in order to obtain a different set of methylated regions from the enriched first native cfDNA treatment group. In some cases, the second native cfDNA treatment group, which results in the first input control, can be about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, about 30%, about 35%, about 40%, about 45%, about 50%, or more of the original native cfDNA sample.

In some cases, sheared DNA samples are split into two treatment groups prior to MeDIP enrichment. The first sheared DNA treatment group can be enriched as described above. The second sheared DNA treatment group can be used as a second input control in order to obtain a different set of methylated regions from the enriched first sheared DNA treatment group. In some cases, the second sheared DNA treatment group, which results in the second input control, can be about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, about 30%, about 35%, about 40%, about 45%, about 50%, or more of the original sheared DNA sample.

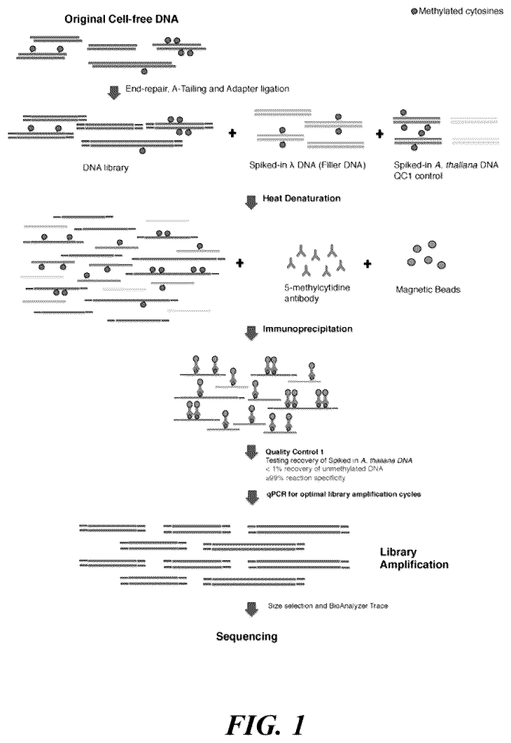

After the step of incubation with CpG methyltransferase, one of the two native cfDNA treatment groups can be prepared to undergo cfMeDIP-seq, as illustrated in FIG. 1. For example, one of the two native cfDNA treatment groups can be supplemented with spike-in control DNA, undergo end-pairing, A-tailing, adapter ligation, and/or other preparation thereof to permit sequencing. The one group can further undergo immunoprecipitation by pulling down the hypermethylated regions with a CpG methylated binder. Filler DNAs as disclosed herein can also be added to the one group of the native cfDNA treatment groups. The hypermethylated regions of the one group of native cfDNAs are enriched. The other group, which serves as the input control does not go through the hypermethylated region enrichment. The native cfDNAs with hypermethylated regions enriched can undergo cfMeDIP-seq.

After the step of incubation with CpG methyltransferase, one of the two sheared cfDNA treatment groups can be prepared to undergo cfMeDIP-seq, as illustrated in FIG. 1. For example, one of the two sheared cfDNA treatment groups can be supplemented with spike-in control DNA, undergo end-pairing, A-tailing, adapter ligation, and/or other preparation thereof to permit sequencing. The one group can further undergo immunoprecipitation by pulling down the hypermethylated regions with a CpG methylated binder. Filler DNAs as disclosed herein can also be added to the one group of the sheared cfDNA treatment groups. The hypermethylated regions of the one group of sheared cfDNAs are enriched. The other group, which serves as the input control does not go through the hypermethylated region enrichment. The sheared cfDNAs with hypermethylated regions enriched can undergo cfMeDIP-seq.

In some cases, the first and/or second input controls are processed through incubation and amplification. Alternatively, in some cases, the first and/or second input controls are not processed through incubation and amplification.

Hypermethylated nucleic acids and/or input controls can be sequenced (e.g., using cfMeDIP-seq) or prepared for sequencing (e.g., creation of sequencing reads). Sequencing can occur before combination of hypermethylated DNA sample sequences and hypermethylated sheared DNA sequences. Alternatively, sequencing can occur after combination of hypermethylated DNA sample sequences and hypermethylated sheared DNA sequences.

Sequencing results and/or sequencing reads can be compared. Data analysis of the sequencing results and/or sequencing reads of the native cfDNA sample, the first input control, the sheared DNA sample, and the second input control generates high methylation signal peaks (corresponds to hypermethylated regions). The high methylation signal peaks of each enriched group aforementioned is normalized against methylation signals generated from its input control. The different methylation signal peaks, i.e., a set of different genomic regions between the enriched native cfDNA and its input control is collated with the different methylation signal peaks, i.e., a set of different genomic regions between the enriched cfDNA mimic and its input control to form a library of sequencing reads. This library of sequencing reads may be used as a new background profile (i.e., contains about 1,218,413 genomic window of 300 bp) instead of a whole genome profile (i.e., contains about 9,583,339 genomic window of 300 bp) for further processing other sequence reads obtained from cfDNA samples obtained from cancer patients, patients at risk of developing cancer, patients suspected of having cancer, caner patients before and after treatment directed to cure the cancer, and healthy control subjects. A background profile can be used to compare healthy and cancerous cell-free nucleic acid samples to determine a disease state. The use of a background profile can allow for the identification of additional differentially methylated regions between healthy and cancerous cell-free nucleic acid samples.

In some cases, a nucleic acid sample (e.g., a cell-free nucleic acid sample) can be taken from a subject. A nucleic acid sample can be derived from a sample obtained from a subject. Non-limiting examples of samples that can be obtained from a subject can include blood, urine, saliva, cerebrospinal fluid, ascites or peritoneal fluid, pleural effusion, pericardial effusion, synovial fluid, gastric juice, and breast milk. A subject can be healthy or can have or be suspected of having a disease or condition. A nucleic acid sample from a subject can be processed in the same way as a cfDNA sample as described above. In some cases, a nucleic acid sample from a subject can be sequenced in order to generate sequencing reads of enriched methylated DNA regions. In some cases, the sequencing reads of enriched methylated DNA regions derived from a sample from a subject can be used to generate a first plurality of differentially methylated regions (DMRs). The first plurality of DMRs can be mapped to the background region. Alternatively or in addition to, the sequencing reads of enriched methylated DNA regions derived from a sample from a subject can be used to generate a second plurality of differentially methylated regions (DMRs). The second plurality of DMRs can be mapped to a whole genome. A DMR can comprise hypermethylation or hypomethylation. The first plurality of DMRs can be greater than the second plurality of DMRs. Alternatively, the second plurality of DMRs can be greater than the first plurality of DMRs. Alternatively the first plurality of DMRs and the second plurality of DMRs can comprise the same number of DMRs. In some cases, using the first plurality of DMRs to classify a sample as diseased or healthy comprises increased separation between diseases and healthy samples as would occur if the second plurality of DMRs was used to classify a sample as diseased or healthy.

In some cases, a background region can be used distinguish between healthy and cancerous cell-free nucleic acid samples. In some cases, a background region can be used to identify DMRs from healthy and/or cancerous cell-free nucleic acid samples. In some cases, the DMRs identified by using the background region comprises using the DMRs identified by using genome-wide background. In some cases, the DMRs identified by using background region comprises a subset of the DMRs identified by using genome-wide background. In some cases, the DMRs identified by using a background region identify more DMRs as compared to using genome-wide background.

In some cases, the use of a background region can identify more than at least about 100, at least about 150, at least about 200, at least about 250, at least about 300, at least about 350, at least about 400, at least about 450, at least about 500, at least about 550, at least about 600, at least about 650, at least about 700, at least about 750, at least about 800, at least about 850, at least about 900, at least about 1000, or more DMRs compared to using genome-wide background. In some cases, the use of a background region can identify more than at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least 45-fold, at least about 50-fold, at least about 100-fold, at least about 150-fold, at least about 200-fold, at least about 250-fold, at least about 300-fold, at least about 350-fold, at least about 400-fold, at least about 450-fold, at least about 500-fold, or more DMRs compared to using genome-wide background.

In some cases, the use of a background region reduce the run time to identify DMRs by at least about 5 minutes, at least about 10 minutes, at least about 15 minutes, at least about 20 minutes, at least about 25 minutes, at least about 30 minutes, at least about 35 minutes, at least about 40 minutes, at least about 45 minutes, at least about 50 minutes, at least about 55 minutes, at least about 1 hour, at least about 1.5 hours, at least about 2 hours, at least about 2.5 hours, at least about 3 hours, at least about 3.5 hours, at least about 4 hours, at least about 4.5 hours, at least about 5 hours, at least about 5.5 hours, at least about 6 hours, at least about 6.5 hours, at least about 7 hours, at least about 7.5 hours, at least about 8 hours, at least about 8.5 hours, at least about 9 hours, at least about 9.5 hours, at least about 10 hours, or more as compared to using genome-wide background to identify DMRs.

In some cases, the use of a background region reduce the run time to identify DMRs by about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least 45-fold, at least about 50-fold, or more as compared to using genome-wide background to identify DMRs.

In some cases, hypermethylation, the process by which methyl groups are added to nucleic acid molecules such as DNA, can identify regions that are most likely to be depleted using a binder specific for methylated DNA (e.g., 5mC antibodies). This identification can create a validated set of genomic regions which can more accurately distinguish between cancer and healthy samples in cfDNA. A hypermethylated section of DNA may be methylated at least 50%, at least 55%, at least 60%, at least 65%, at least 70%, at least 75%, at least 80%, at least 85%, at least 90%, at least 95%, or more across all bases. A hypermethylated section of DNA may be tightly packed, resulting in a silenced gene.

Depletion of all or a portion of the methylated nucleic acid molecules of a plurality of nucleic acid molecules of a biological sample may comprise contacting the methylated nucleic acid molecules with a binder (e.g., an affinity molecule, such as an antibody or a protein, specific to methylated nucleotide residues). For example, creation of a depleted sequencing library can comprise contacting a plurality of nucleic acid molecules (e.g., cfDNA molecules) or amplicons thereof with a binder selective for a methylated region of nucleic acid molecules (e.g., a methylcytosine binder (MBD), such as an MBD-Fc fusion protein). In some cases, a binder may be specific to one or more methylated nucleotide species (e.g., 5-methylcytosine (5mC)), for instance, as shown in FIG. 1. Cell-free Methylated DNA ImmunoPrecipitation sequencing (cfMeDIP-seq), a genome-wide molecular profiling technique, can enrich for methylated cfDNA fragments through use of a binder, such as an anti-5-methylcytosine (anti-5mC) antibody or methyl-CpG-binding domain (MBD) protein (e.g., MBD-Fc fusion proteins). As described herein, cfMeDIP-seq can comprise a portion of methods and systems for depleting a cfDNA sample of methylated DNA fragments, leaving behind hypomethylated or unmethylated cfDNA fragments, such as ctDNA. Thus, the identification of hypomethylated or unmethylated cell-free DNA within a clinical sample may be useful in determining the presence of a tumor or cancer in a subject.

In some cases, depletion of a plurality of nucleic acid molecules (e.g., in the creation of a depleted sequencing library and/or the determination of a presence or sequence identity of a nucleic acid molecule) may comprise removing one or more nucleic acid molecules having a methylation level above a threshold methylation level. (e.g., wherein the one or more removed nucleic acid molecules are hypermethylated, for instance, relative to one or more nucleic acid molecules not removed during depletion). In some cases, a methylation level of a particular nucleic acid fragments (e.g., DNA fragments) may be considered to reach the threshold methylation level when a binder with a sufficient specificity for methylated cytosines is able to bind to the particular nucleic acid fragments either with or without using filler DNA as described here. In some cases, a methylation level of particular nucleic acid fragments (e.g., DNA fragments) may be considered to be below the threshold methylation level when a binder with a sufficient specificity for methylated cytosines is not able to bind to the particular nucleic acid fragments either with or without using filler DNA, as described here. In some cases, depletion of a plurality of nucleic acid molecules (e.g., in the creation of a depleted sequencing library and/or the determination of a presence or sequence of a nucleic acid molecule) results in (e.g., provides) a remainder population of the plurality of nucleic acid molecules, wherein the remainder of the plurality of nucleic acid molecules comprises (or, in some cases, consists of) nucleic acid molecules having a methylation level below the threshold methylation level (e.g., wherein the remainder population is hypomethylated/unmethylated relative to one or more nucleic acid molecules removed from the plurality of nucleic acid molecules during depletion). A methylation level may be calculated as a percentage of hypermethylated nucleic acid fragments compared to all the nucleic acid fragments contained in a sample. In some cases, a threshold methylation level can be from 0.1% to 1%, 1% to 5%, 5% to 10%, 10% to 15%, 15% to 20%, 20% to 25%, 25% to 30%, 30% to 35%, 35% to 40%, 40% to 45%, 45% to 50%, 50% to 55%, 55% to 60%, 65% to 70%, 70% to 75%, 75% to 80%, 80% to 85%, 85% to 90%, 95% to 100%, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at most 1%, at most 5%, at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95%, or at most 100%.

In some cases, the creation of a subset of the global or whole-genome defines a background for methylation assays. A background can comprise a plurality of genomic windows or window regions that can be defined by their specificity to a binder. Alternatively or in addition to, a background can comprise a plurality of genomic windows or window regions that are determined through specific methylation patterns. A background can comprise at least 1, at least 2, at least 3, at least 4, at least 5, at least 6, at least 7, at least 8, at least 9, at least 10, at least 15, at least 20, at least 30, at least 40, at least 50, at least 60, at least 70, at least 80, at least 90, at least 100, at least 200, at least 300, at least 400, at least 500, at least 600, at least 700, at least 800, at least 900, at least 1,000, at least 5,000, at least 10,000, at least 50,000, or more genomic windows or window regions.

A genomic window or window region can have a length of at least about 10, at least about 20, at least about 30, at least about 40, at least about 50, at least about 60, at least about 70, at least about 80, at least about 90, at least about 100, at least about 150, at least about 200, at least about 250, at least about 300, at least about 350, at least about 400, at least about 450, at least about 500, at least about 550, at least about 600, at least about 650, at least about 700, at least about 750, at least about 800, at least about 850, at least about 900, at least about 950, at least about 1,000, at least about 2,000, at least about 3,000, at least about 4,000, at least about 5,000, at least about 6,000, at least about 7,000, at least about 8,000, at least about 9,000, at least about 10,000, at least about 20,000, at least about 30,000, at least about 40,000, at least about 50,000, or more base pairs (bp). In some cases, a genomic window is about 300 bp in length. In some cases, the genomic windows are adjacent to one another in the genome. Alternatively or in addition to, the genomic windows can be non-adjacent regions on the genome.

In some cases, a first plurality of nucleic acid molecules (e.g., comprising nucleic acid molecules, such as cfDNA, from a biological sample of a subject) may be combined (e.g., mixed) with a second plurality of nucleic acid molecules (e.g., wherein the second plurality of nucleic acid molecules is not from the subject from whom the biological sample was taken), for instance, as shown in FIG. 1. In some cases, the second plurality of nucleic acid molecules comprises supplemental processed DNA (e.g., comprising 1 DNA). In some cases, each of the second plurality of nucleic acid molecules does not align to a human genome.

In some cases, a method or system disclosed herein may comprise determining or identifying a sequence of all or a portion of a depleted nucleic acid molecule population (e.g., remainder population of a plurality of nucleic acid fragments of a biological sample after pulling down hypermethylated nucleic acid fragments), for example, using a sequencer (e.g., as shown in FIG. 1). In some cases, a remainder population of nucleic acid molecules may be purified (e.g., after library creation) to yield a plurality of purified nucleic acid molecules, for example, prior to or as part of a process of determining or identifying a sequence of all or a portion of the depleted nucleic acid molecule population. In some cases, all or a portion of the plurality of purified nucleic acid molecules may be amplified (e.g., via polymerase chain reaction), for instance, prior to or as part of a process of determining or identifying a sequence of all or a portion of the depleted nucleic acid molecule population. In some cases, a population of amplified nucleic acid molecules or a derivative thereof (e.g., comprising amplicons of all or a portion of the plurality of purified nucleic acid molecules) may be subjected to sequencing (e.g., for the determination and/or identification of a sequence of the nucleic acid molecules). In some cases, the sequencing may be achieved using a sequencer, as described herein. In some cases, a sequence of a plurality of nucleic acid molecules of a biological sample (or a derivative thereof) may be identified or determined using an array or polymerase chain reaction. In some cases, the presence of a tumor-derived nucleic acid molecule may be determined by calculating a sum of reads per kilobase per million (RPKM) for a region of the genome (e.g., all or a portion of the genome, such as just CpG islands or just CpG island shores). In some cases, the presence of a tumor-derived nucleic acid molecule may be indicated when a depleted sequencing library (e.g., comprising a remainder population of nucleic acids) is observed to have a low sum of RPKMs, e.g., lower than 70,000, lower than 60,000, lower than 50,000, lower than 40,000, or lower than 30,000 across one or more regions of interest (e.g., CpG islands or CpG island shores).

### Supplemental Processed DNA (filler DNA)

In some cases, supplemental processed DNA (e.g., filler DNA) may be added to a first plurality of nucleic acids (e.g., a plurality of nucleic acids from a biological sample, which may comprise cfDNA from healthy tissue and/or cfDNA from tumor tissue, such as ctDNA), for instance as shown in FIG. 1. In some cases, addition of supplemental processed DNA (e.g., a second plurality of nucleic acid molecules) to a first plurality of nucleic acid molecules can increase the specificity and/or sensitivity of a method, or system described herein, for instance, with respect to the detection and/or identification of a nucleic acid sequence of the first plurality of nucleic acid molecules. In some cases, addition of supplemental processed DNA (e.g., a second plurality of nucleic acid molecules) to a first plurality of nucleic acid molecules may increase the rate of depletion of a methylated region of a nucleic acid sequence, e.g., during the practice of some embodiments of methods and systems described herein. In some cases, addition of supplemental processed DNA (e.g., a second plurality of nucleic acid molecules) to a first plurality of nucleic acid molecules (e.g., comprising cfDNA of a biological sample) may increase a binder's selectivity for one or more (e.g., a plurality of) methylated regions of the first plurality of nucleic acid molecules. In some cases, supplemental processed DNA (e.g., the second plurality of nucleic acid molecules) may be added to the first plurality of nucleic acid molecules in an amount sufficient to bring the combined mixture of nucleic acid molecules to a desired total mass. In some cases, a desired total mass for use in a method or system described herein can be from 20 ng to 30 ng, from 30 ng to 40 ng, from 40 ng to 50 ng, from 50 ng to 60 ng, from 60 ng to 70 ng, from 70 ng to 80 ng, from 80 ng to 90 ng, from 90 ng to 100 ng, from 100 ng to 110 ng, from 110 ng to 120 ng, from 120 ng to 130 ng, from 130 ng to 140 ng, from 140 ng to 150 ng, from 150 ng to 160 ng, from 160 ng to 170 ng, from 170 ng to 180 ng, from 180 ng to 190 ng, from 190 ng to 200 ng, greater than 200 ng, or less than 20 ng. In some cases, an amount of supplemental processed DNA from 1 ng to 5 ng, from 5 ng to 10 ng, from 10 ng to 20 ng, from 20 ng to 30 ng, from 30 ng to 40 ng, from 40 ng to 50 ng, from 50 ng to 60 ng, from 60 ng to 70 ng, from 70 ng to 80 ng, from 80 ng to 90 ng, from 90 ng to 100 ng, from 100 ng to 110 ng, from 110 ng to 120 ng, from 120 ng to 130 ng, from 130 ng to 140 ng, from 140 ng to 150 ng, from 150 ng to 160 ng, from 160 ng to 170 ng, from 170 ng to 180 ng, from 180 ng to 190 ng, from 190 ng to 200 ng, greater than 200 ng, less than 20 ng, less than 10 ng, or less than 5 ng can be added to a first plurality of nucleic acid molecules (e.g., to bring the total mixture of the supplemental processed DNA and the first plurality of nucleic acid molecules to the desired total mass). In some embodiments, the present disclosure comprises methods and systems for filling in the sample with an amount of supplemental processed DNA (e.g., filler DNA) to generate a mixture sample, wherein the mixture sample comprises at least about 50 ng, 55 ng, 60 ng, 65 ng, 70 ng, 75 ng, 80 ng, 85 ng, 90 ng, 95 ng, 100 ng, 120 ng, 140 ng, 160 ng, 180 ng, 200 ng, or any amount in between the numbers of the total amount of the nucleic acid mixture. In some embodiments, the supplemental processed DNA comprises at least about 5%, 10%, 15%, 20%, 30%, 40%, 50%, 60%, 70%, 80%, 90%, or 100% methylated supplemental processed DNA with remainder being unmethylated supplemental processed DNA, in some cases between 5% and 50%, or between 10%-40%, or between 15%-30% methylated supplemental processed DNA. In some embodiments, the mixture sample comprise an amount of supplemental processed DNA from about 20 ng to about 100 ng, about 30 ng to about 100 ng, or about 50 ng to about 100 ng. In some embodiments, the cell-free DNA from the sample and the first amount of supplemental processed DNA together comprises at least 50 ng of total DNA. In some embodiments, the cfDNA from the sample and the first amount of supplemental processed DNA together comprises at least 100 ng of total DNA.

In some cases, supplemental processed DNA may be produced by fragmentation (e.g., via sonication). In some embodiments, the supplemental processed DNA may be about 50 bp to about 800 bp long, about 100 bp to about 600 bp long, or about 200 bp to about 600 bp long. In some embodiments, the supplemental processed DNA is double stranded. The supplemental processed DNA may be double stranded DNA. For example, the supplemental processed DNA may be junk DNA. The supplemental processed DNA may also be endogenous or exogenous DNA. For example, the supplemental processed DNA is non-human DNA, such as 2. DNA. As used herein, “A DNA” refers to Enterobacteria phage 2 DNA. In some embodiments, the supplemental processed DNA has no alignment to human DNA. In some embodiments, supplemental DNA can be hypermethylated.

### Samples

A sample can be any biological sample isolated from a subject. For example, a sample may comprise, without limitation, bodily fluid, whole blood, platelets, serum, plasma, stool, white blood cells or leukocytes, endothelial cells, tissue biopsies, synovial fluid, lymphatic fluid, ascites fluid, interstitial or extracellular fluid, the fluid in spaces between cells, including gingival crevicular fluid, bone marrow, cerebrospinal fluid, saliva, mucous, sputum, semen, sweat, urine, fluid from nasal brushings, fluid from a pap smear, or any other bodily fluids. A bodily fluid may include saliva, blood, or serum. A sample may also be a tumor sample, which may be obtained from a subject by various approaches, including, but not limited to, venipuncture, excretion, ejaculation, massage, biopsy, needle aspirate, lavage, scraping, surgical incision, or intervention or other approaches. A sample may be a cell-free sample (e.g., substantially free of cells). DNA samples may be denatured, for example, using sufficient heat.

The sample may be taken from a subject with a disease or disorder. The sample may be taken from a subject suspected of having a disease or a disorder. In some embodiments, the sample may be obtained before and/or after treatment of a subject with a disease or disorder. Samples may be obtained from a subject during a treatment or a treatment regime. Multiple samples may be obtained from a subject to monitor the effects of the treatment over time. The disease or disorder may be a cancer. A cancer can be a late-stage or an early-stage cancer. Specific exemplary examples of cancer types include suitable for detection with the methods according to the disclosure include acute lymphoblastic leukemia, acute myeloid leukemia, adrenocortical carcinoma, AIDS-related cancers, AIDS-related lymphoma, anal cancer, appendix cancer, astrocytomas, basal cell carcinoma, bile duct cancer, bladder cancer, bone cancers, brain tumors, such as cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas, Burkitt lymphoma, carcinoma of unknown primary origin, central nervous system lymphoma, cerebellar astrocytoma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma, germ cell tumors, gallbladder cancer, gastric cancer, gastrointestinal carcinoid tumor, gastrointestinal stromal tumor, gliomas, hairy cell leukemia, head and neck cancer, heart cancer, hepatocellular (liver) cancer, Hodgkin lymphoma, Hypopharyngeal cancer, intraocular melanoma, islet cell carcinoma, Kaposi sarcoma, kidney cancer, laryngeal cancer, lip and oral cavity cancer, liposarcoma, liver cancer, lung cancers, such as non-small cell and small cell lung cancer, lymphomas, leukemias, macroglobulinemia, malignant fibrous histiocytoma of bone/osteosarcoma, medulloblastoma, melanomas, mesothelioma, metastatic squamous neck cancer with occult primary, mouth cancer, multiple endocrine neoplasia syndrome, myelodysplastic syndromes, myeloid leukemia, nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer, ovarian germ cell tumor, pancreatic cancer, pancreatic cancer islet cell, paranasal sinus and nasal cavity cancer, parathyroid cancer, penile cancer, pharyngeal cancer, pheochromocytoma, pineal astrocytoma, pineal germinoma, pituitary adenoma, pleuropulmonary blastoma, plasma cell neoplasia, primary central nervous system lymphoma, prostate cancer, rectal cancer, renal cell carcinoma, renal pelvis and ureter transitional cell cancer, retinoblastoma, rhabdomyosarcoma, salivary gland cancer, sarcomas, skin cancers, skin carcinoma merkel cell, small intestine cancer, soft tissue sarcoma, squamous cell carcinoma, stomach cancer, T-cell lymphoma, throat cancer, thymoma, thymic carcinoma, thyroid cancer, trophoblastic tumor (gestational), cancers of unknown primary site, urethral cancer, uterine sarcoma, vaginal cancer, vulvar cancer, Waldenström macroglobulinemia, and Wilm's tumor. In an embodiment, the cancer is head and neck squamous cell carcinoma.

The sample may be taken from a healthy individual. In some cases, samples may be taken longitudinally from the same individual. In some cases, samples acquired longitudinally may be analyzed with the goal of monitoring individual health and early detection of health issues. In some embodiments, the sample may be collected at a home setting or at a point-of-care setting and subsequently transported by a mail delivery, courier delivery, or other transport method prior to analysis. For example, a home user may collect a blood spot sample through a finger prick, which blood spot sample may be dried and subsequently transported by mail delivery prior to analysis. In some cases, samples acquired longitudinally may be used to monitor response to stimuli expected to impact healthy, athletic performance, or cognitive performance. Non-limiting examples include response to medication, dieting, or an exercise regimen.

In some embodiments, the present disclosure provides a system, method, or kit that includes or uses one or more biological samples. The one or more samples used herein may comprise any substance containing or presumed to contain nucleic acids. A sample may include a biological sample obtained from a subject. In some embodiments, a biological sample is a liquid sample.

In some embodiments, the sample comprises less than about 100 ng, 90 ng, 80 ng, 75 ng, 70 ng, 60 ng, 50 ng, 40 ng, 30 ng, 20 ng, 10 ng, 5 ng, 1 ng or any amount in between the numbers of cell-free nucleic acid molecules. Further, in some embodiments, the sample comprises less than about 1 pg, less than about 5 pg, less than about 10 pg, less than about 20 pg, less than about 30 pg, less than about 40 pg, less than about 50 pg, less than about 100 pg, less than about 200 pg, less than about 500 pg, less than about 1 ng, less than about 5 ng, less than about 10 ng, less than about 20 ng, less than about 30 ng, less than about 40 ng, less than about 50 ng, less than about 100 ng, less than about 200 ng, less than about 500 ng, less than about 1000 ng, or any amount in between the numbers of cell-free nucleic acid molecules.

In some cases, creation or provision of a plurality of nucleic acid molecules from a biological sample can comprise performing one or more of end-repair, A-tailing, and/or adapter ligation on the plurality of nucleic acid molecules (e.g., after purification from the biological sample).

In some embodiments, a sample may be taken at a first time point and sequenced, and then another sample may be taken at a subsequent time point and sequenced. Such methods may be used, for example, for longitudinal monitoring purposes to track the development or progression of a disease. In some embodiments, the progression of a disease may be tracked before treatment, after treatment, or during the course of treatment, to determine the treatment's effectiveness. For example, a method as described herein may be performed on a subject prior to, and after, a medical treatment to measure the disease's progression or regression in response to the medical treatment.

After obtaining a sample from the subject, the sample may be processed to generate datasets indicative of a disease or disorder of the subject. For example, a presence, absence, or quantitative assessment of cell-free nucleic acid molecules (e.g., ctDNA molecules) of the sample at a panel of cancer-associated genomic loci or microbiome-associated loci may be indicative of a cancer of the subject. Processing the sample obtained from the subject may comprise (i) subjecting the sample to conditions that are sufficient to isolate, enrich, or extract a plurality of cell-free nucleic acid molecules, and (ii) assaying the plurality of cell-free nucleic acid molecules to generate the dataset (e.g., nucleic acid sequences). In some embodiments, a plurality of cell-free nucleic acid molecules is extracted from the sample and subjected to sequencing to generate a plurality of sequencing reads.

In some embodiments, the cell-free nucleic acid molecules may comprise cell-free ribonucleic acid (cfRNA) or cell-free deoxyribonucleic acid (cfDNA). The cell-free nucleic acid molecules (e.g., cfRNA or cfDNA) may be extracted from the sample by a variety of methods. The cell-free nucleic acid molecule may be enriched by a plurality of probes configured to enrich nucleic acid (e.g., RNA or DNA) molecules corresponding to a panel of cancer-associated genomic loci. The probes may have sequence complementarity with nucleic acid sequences from one or more of the genomic loci of the panel of cancer-associated genomic loci. The panel of cancer-associated genomic loci may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or more distinct cancer-associated genomic loci. The probes may be nucleic acid molecules (e.g., RNA or DNA) having sequence complementarity with nucleic acid sequences (e.g., RNA or DNA) of the one or more genomic loci (e.g., cancer-associated genomic loci). These nucleic acid molecules may be primers or enrichment sequences. The assaying of the sample using probes that are selective for the one or more genomic loci (e.g., cancer-associated genomic loci or microbiome-associated loci) may comprise use of array hybridization, polymerase chain reaction (PCR), or nucleic acid sequencing (e.g., RNA sequencing or DNA sequencing).

Certain methods of capturing cell-free methylated DNA are described in WO 2017/190215 and WO 2019/010564, both of which are incorporated by reference in their entireties and for all purposes.

### Nucleic Acid Molecule Sequencing

Various assays may be used in methods of the present disclosure, such as library preparation (which may include polymerase chain reaction (PCR)) followed by sequencing (e.g., next-generation sequencing, Sanger sequencing, etc.). Next-generation sequencing (NGS) techniques, also known as high-throughput sequencing, may include various sequencing technologies including: Illumina (Solexa) sequencing, Roche 454 sequencing, Ion torrent: Proton/PGM sequencing, SOLID sequencing, long reads sequencing (Oxford Nanopore and Pactbio). NGS allow for the sequencing of DNA and RNA much more quickly and cheaply than the previously used Sanger sequencing. In some embodiments, said sequencing is optimized for short read sequencing.

Sequencing libraries that are hypermethylated may improve the specificity, the sensitivity, and/or the efficiency of methods and systems for processing nucleic acids. For example, hypermethylated sequencing libraries may improve the specificity, the sensitivity, and/or the efficiency of assays for determining the presence and/or sequence identity of a nucleic acid sequence. A hypermethylated sequencing library may comprise a plurality of nucleic acids and/or fragments thereof. In some cases, a hypermethylated sequencing library may comprise a plurality of nucleic acid molecules (e.g., a population of nucleic acids and/or fragments thereof). The plurality of nucleic acid molecules may comprise all or a portion of a first plurality of nucleic acid molecules, e.g., wherein the first plurality of nucleic acid molecules comprises one or more nucleic acid molecules that comprise a methylated nucleic acid residue and one or more nucleic acid molecules that does not comprise a methylated nucleic acid residue. In some cases, a methylated nucleic acid may comprise one or more methylated nucleic acid residues. For instance, a methylated nucleic acid may comprise one or more methylated cytosines (e.g., one or more 5-methylcytosines (5mC) and/or one or more 5-hydroxymethylcytosines (5hmC)).

A plurality of nucleic acid molecules (e.g., a plurality of nucleic acid molecules derived from a biological sample) may be hypermethylated and enriched by using a binder, e.g., as described herein, to form a hypermethylated sequencing library which can be used as a background as opposed to a whole-genome background for use in analysis of cfDNA. In some cases, DNA may be hypermethylated before use of a binder to create a sequencing library with a background. The background sequencing library may comprise a set of background genomic regions that are enriched by the binder.

The present disclosure provides methods and technologies for determining the sequence of nucleotide bases in one or more polynucleotides. The polynucleotides may be, for example, nucleic acid molecules such as deoxyribonucleic acid (DNA) or ribonucleic acid (RNA), including variants or derivatives thereof (e.g., single stranded DNA). Sequencing may be performed by various systems currently available, such as, without limitation, a sequencing system by Illumina®, Pacific Biosciences (PacBio®), Oxford Nanopore®, or Life Technologies (Ion Torrent®). Further, any sequencing methods that provide fragment length such as paired-end sequencing may be utilized. Alternatively or additionally, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

In some embodiments, the sequencing reads are obtained via a next-generation sequencing method or a next-next-generation sequencing method. In some embodiments, the sequencing methods comprise cfMeDIP sequencing, e.g., comprising steps or systems as described by Shen et al., (“Sensitive tumor detection and classification using plasma cell-free DNA methylomes,” (2018) Nature), which is incorporated herein in its entirety. In some embodiments, sequencing can be performed using methyl-CpG-binding domain sequencing (MBD-seq). In some cases, MBD-seq can comprise capture (e.g., via a binder, such as an antibody specific to a species of methylated nucleotide) of double-stranded, methylated DNA fragments for sequencing of methylation-enriched DNA fragment libraries. In some embodiments, the sequencing methods comprise CAncer Personalized Profiling by deep Sequencing (CAPP-Seq), which is a next-generation sequencing based method used to quantify circulating DNA in cancer (ctDNA). In some embodiments, the sequencing methods comprise chromatin immunoprecipitation sequencing (ChIP-Seq). This method may be generalized for any cancer type that is known to have recurrent mutations and may detect one molecule of mutant DNA in 10,000 molecules of healthy DNA. In some embodiments, the sequencing comprises bisulfite sequencing. Alternatively, in some embodiments, the sequencing does not comprise bisulfite sequencing.

Sequencing can comprise analysis of the results of sequencing methods. In some embodiments, sequencing analysis can comprise using Model-based Analysis for ChIP-Seq (MACS) software. The MACS algorithm captures the influence of genome complexity to evaluate the significance of enriched regions. Sequencing analysis can comprise identifying broad peaks (broad peak calling) or identifying narrow peaks (narrow peak calling). In some cases, hypermethylated regions can be identified using narrow peak calling. Peak annotations that note regions of interest can be produced by the MACS algorithm by determining signals that differ significantly between two samples (e.g., between a sample and the background region). In some cases, hypermethylated regions can be identified using broad peak calling. In some cases, both narrow peak calling and broad peak calling will identify the same hypermethylated regions. In some cases, narrow peak calling and broad peak calling will identify different hypermethylated regions. Additional processing of peak annotations can merge regions of interest across multiple samples to result in higher resolution and more accurate results. More accurate results can comprise the inclusion of regions that have very few reads in samples, but which can be leveraged to differentiate between healthy and disease samples. In some cases, sequencing analysis can be illustrated using a gene heatmap. Alternatively or in addition to, sequencing analysis can be illustrated using a uniform manifold approximation and projection (UMAP) plot.

In some cases, a sample or portion thereof (e.g., a plurality of nucleic acids of a sample) may be subjected to library preparation before sequencing. In short, after end-repair and A-tailing, the samples are ligated to nucleic acid adapters and digested using enzymes.

In some embodiments, sequencing comprises modification of a nucleic acid molecule or fragment thereof, for example, by ligating a barcode, a unique molecular identifier (UMI), or another tag to the nucleic acid molecule or fragment thereof. Ligating a barcode, UMI, or tag to one end of a nucleic acid molecule or fragment thereof may facilitate analysis of the nucleic acid molecule or fragment thereof following sequencing. In some embodiments, a barcode is a unique barcode (e.g., a UMI). In some embodiments, a barcode is non-unique, and barcode sequences may be used in connection with endogenous sequence information such as the start and stop sequences of a target nucleic acid (e.g., the target nucleic acid is flanked by the barcode and the barcode sequences, in connection with the sequences at the beginning and end of the target nucleic acid, creates a uniquely tagged molecule). A barcode, UMI, or tag may be a known sequence used to associate a polynucleotide or fragment thereof with an input or target nucleic acid molecule or fragment thereof. A barcode, UMI, or tag may comprise natural nucleotides or non-natural (e.g., modified) nucleotides (e.g., as described herein). A barcode sequence may be contained within an adapter sequence such that the barcode sequence may be contained within a sequencing read. A barcode sequence may comprise at least 4, 5, 6, 7, 8, 9, 10, 11, 12, 13, 14, 15, 16, or more nucleotides in length. In some cases, a barcode sequence may be of sufficient length and may be sufficiently different from another barcode sequence to allow the identification of a sample based on a barcode sequence with which it is associated. A barcode sequence, or a combination of barcode sequences, may be used to tag and subsequently identify an “original” nucleic acid molecule or fragment thereof (e.g., a nucleic acid molecule or fragment thereof present in a sample from a subject). In some cases, a barcode sequence, or a combination of barcode sequences, is used in conjunction with endogenous sequence information to identify an original nucleic acid molecule or fragment thereof. For example, a barcode sequence, or a combination of barcode sequences, may be used with endogenous sequences adjacent to a barcode, UMI, or tag (e.g., the beginning and end of the endogenous sequences).

As described herein, the prepared libraries may be combined with filler nucleic acids (e.g., filler) DNAs) to minimize the effect of low abundance ctDNA in the prepared libraries and generate mixed samples. In some embodiments, when the disease and/or condition is a locoregionally (non-metastatic) cancer, the amount of ctDNA is low and may not be easily and accurately measured and quantified. The mixed samples are brought to at least about 50 ng, 80 ng, 100 ng, 120 ng, 150 ng, or 200 ng and are subjected to further enrichment.

Processing a nucleic acid molecule or fragment thereof may comprise performing nucleic acid amplification. For example, any type of nucleic acid amplification reaction may be used to amplify a target nucleic acid molecule or fragment thereof and generate an amplified product. Non-limiting examples of nucleic acid amplification methods include reverse transcription, primer extension, polymerase chain reaction (PCR), ligase chain reaction, asymmetric amplification, rolling circle amplification, and multiple displacement amplification (MDA). Examples of PCR include, but are not limited to, quantitative PCR, real-time PCR, digital PCR, emulsion PCR, hot start PCR, multiplex PCR, asymmetric PCR, nested PCR, and assembly PCR. Nucleic acid amplification may involve one or more reagents such as one or more primers, probes, polymerases, buffers, enzymes, and deoxyribonucleotides. Nucleic acid amplification may be isothermal or may comprise thermal cycling. and/or with the length of the endogenous sequence.

### Binders

A binder may be used to deplete a population of nucleic acid molecules (e.g., a plurality of nucleic acid molecules derived from a biological sample). In some cases, a binder can be used to deplete a plurality of nucleic acid molecules of one or more nucleic acid molecules having a methylation level at or above a threshold methylation level (e.g., by binding to one or more methylated nucleotides of the one or more nucleic acid molecules). A binder may be used to enrich a population of nucleic acid molecules (e.g., a plurality of nucleic acids derived from a biological sample). In some cases, a binder can exhibit a reduced level of non-specific binding to non-methylated nucleotides or non-methylated genomic regions.

In some cases, a binder can be specific to one or more methylated nucleotide species (e.g., 5-methylcytosine (5mC), 5-hydroxymethylcytosine (5hmC), 4-methylcytosine (4mC), or 6-methyladenine (6mA)). In some cases, a binder can be selected from the group consisting of an anti-5-methylcytosine antibody or a derivative thereof, an anti-5-carboxylcytosine antibody or a derivative thereof, an anti-5-formylcytosine antibody or a derivative thereof, an anti-5-hydroxymethylcytosine antibody or a derivative thereof, an anti-3-methylcytosine antibody or a derivative thereof, and any combinations thereof. In some cases, the binder can be an anti-5-methylcytosine antibody or a derivative thereof. In some embodiments, the binder is a protein comprising a Methyl-CpG-binding domain. One such exemplary protein is MBD2 protein. As used herein, “Methyl-CpG-binding domain (MBD)” refers to certain domains of proteins and enzymes that is approximately 70 residues long and binds to DNA that contains one or more symmetrically methylated CpGs. The MBD of MeCP2, MBD1, MBD2, MBD4 and BAZ2 mediates binding to DNA, and in cases of MeCP2, MBD1 and MBD2, preferentially to methylated CpG. Human proteins MECP2, MBD1, MBD2, MBD3, and MBD4 comprise a family of nuclear proteins related by the presence in each of a methyl-CpG-binding domain (MBD). Each of these proteins, with the exception of MBD3, is capable of binding specifically to methylated DNA.

In other embodiments, the binder is an antibody and capturing cell-free methylated DNA comprises immunoprecipitating the cell-free methylated DNA using the antibody. As used herein, “immunoprecipitation” refers a technique of precipitating an antigen (such as polypeptides and nucleotides) out of solution using an antibody that specifically binds to that particular antigen. This process may be used to isolate and concentrate a particular protein or DNA from a sample and requires that the antibody be coupled to a solid substrate at some point in the procedure. The solid substrate includes for example beads, such as magnetic beads. Other types of beads and solid substrates may be used.

For example, a 5-mC antibody (e.g., wherein the 5-mC antibody specifically binds to 5-methylcytosine) may be used as a binder. For the immunoprecipitation procedure, in some embodiments at least 0.05 μg of the antibody is added to the sample; Alternatively, at least 0.16 μg of the antibody is added to the sample. In some cases, 0.05 μg to 0.80 μg, 0.16 μg to 0.80 μg, 0.40 μg to 0.80 μg, 0.16 μg to 0.40 μg, 0.10 μg to 0.80 μg, 0.20 μg to 0.60 μg, 0.30 μg to 0.50 μg, or 0.40 μg to 0.50 μg of the antibody can be used. To confirm the immunoprecipitation reaction, in some embodiments the method described herein further comprises the step of adding a second amount of control DNA to the sample.

### Methylation Profile

The present disclosure provides methods and systems for producing a methylation profile of a subject that has a disease and/or condition or is suspected of having such disease and/or condition, wherein the methylation profile may be used to determine whether the subject has the disease and/or condition or is at risk of having the disease and/or condition. In some cases, a methylation profile can comprise analysis (e.g., comprising sequencing) of a plurality of nucleic acids (e.g., a plurality of nucleic acid molecules of a depleted sequencing library, as described herein). In some cases, a methylation profile can comprise detection of methylated nucleotides and/or quantification of methylated nucleotide counts. In some cases, a methylation profile can comprise determination of a methylated signal, e.g., in a population of nucleic acids of a depleted sequencing library, as described herein. In some cases, a methylation profile is compared to a genome-wide background profile. In some cases, a methylation profile is compared to a background profile created using hypermethylated cfDNA.

### Genomic Mutation Profile

The present disclosure provides methods and systems for producing a mutation profile of a subject that has a disease and/or condition or is suspected of having such disease and/or condition, wherein the methylation profile may be used to determine whether the subject has the disease and/or condition or is at risk of having the disease and/or condition. The samples disclosed herein can be subjected to library preparation and next generation deep sequencing, for example to a depth of 1 million (M) to 60 M single reads, 10 M to 60 M single reads, 10M to 100 M single reads, 40 M to 60 M single reads, 40 M to 100 M single reads, 60 M to 100 M single reads, 60 M to 200 M single reads, 1 M to 10 M single reads, 1 M to 40 M single reads, 1 M single reads to 100 M single reads, 1 M single reads to 200 M single reads, at least 1 M single reads, at least 10 M single reads, at least 40 M single reads, at least 60 M single reads, at least 100 M single reads, or at least 200 M single reads. In some cases, sequencing can be performed at low sequencing depth (e.g., 10 M single reads, 20 M single reads, 30 M single reads, 40 M single reads, from 1 M single reads to 10 M single reads, from 10 M single reads to 20 M single reads, from 20 M single reads to 30 M single reads, from 30M single reads to 40 M single reads, at most 10 M single reads, at most 20 M single reads, at most 30 M single reads, or at most 40 M single reads). In some cases, a sample disclosed herein can be subjected to 1 sequencing at a depth of 0.1X to 100X, 0.1X to 60X, 0.1X to 40X, 0.1X to 30X, 0.1X to 20X, 0.1X to 10X, 0.1X to 5.0X, 0.5X to 100X, 0.5X to 60X, 0.5X to 40X, 0.5X to 30X, 0.5X to 20X, 0.5X to 10X, 0.5X to 5.0X, 1.0X to 100X, 1.0X to 60X, 1.0X to 40X, 1.0X to 30X, 1.0X to 20X, 1.0X to 10X, 1.0X to 5.0X, at least 0.1X, at least 0.5X, at least 1.0X, at least 2.0X, at least 3.0X, at least 4.0X, at least 5.0X, at least 10.0X, at least 20.0X, at least 30.0X, at least 40.0X, at least 50.0X, at least 60.0X, at least 100X, at least 200X, at most 0.1X, at most 0.5X, at most 1.0X, at most 2.0X, at most 3.0X, at most 4.0X, at most 5.0X, at most 10.0X, at most 20.0X, at most 30.0X, at most 40.0X, at most 50.0X, at most 60.0X, at most 100X, or at most 200X. A plurality of sequencing reads is generated and analyzed. In some embodiments, deep sequencing may be configured to maximize identifying genomic mutations associated with the disease and/or condition.

In some embodiments, the relative measure of ctDNA abundance is calculated from the mean mutant allele fractions (MAFs). In some embodiments, the mean MAF of mutations identified a subject and comprised in his/her mutation profile ranges from at least about 0.01% to at least about 10%. In some cases, the MAF of a ctDNA fraction of a sample can be about at least 0.01%, 0.02%, 0.03%, 0.04%, 0.05%, 0.06%, 0.07%, 0.08%, 0.09%, 0.1%, 0.15%, 0.2%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, or any percentage in between.

In some embodiments, a generated mutation profile of a subject can be generated from sequencing results. In some embodiments, the mutation profile comprises genetic polymorphisms, such as missense variant, a nonsense variant, a deletion variant, an insertion variant, a duplication variant, an inversion variant, a frameshift variant, or a repeat expansion variant. In some embodiments, the mutation profile may comprise mutation variant derived from a fraction of cell-free nucleic acid molecules of a specific size range. The present disclosure provides methods, systems, and kits for producing a mutation profile of a subject that has a disease and/or condition or is suspected of having such disease and/or condition, wherein the methylation profile may be used to determine whether the subject has the disease and/or condition or is at risk of having the disease and/or condition. Producing a genomic mutation profile can comprise subjecting a plurality of nucleic acid molecules to library preparation and next generation deep sequencing (e.g., MeDIP-seq). A plurality of sequencing reads can be generated and analyzed, and, in some cases, deep sequencing may be configured to maximize identifying genomic mutations associated with the disease and/or condition. For example, a panel of canonical cancer driver genes may be included in a selector for sequencing results analysis. In some embodiments, including genes without known driver effects in a particular cancer type in the analysis of sequencing data may increase the sensitivity of ctDNA detection.

In some embodiments, the relative measure of ctDNA abundance is calculated from the mean mutant allele fractions (MAFs). In some embodiments, the mean MAF of mutations identified a subject and comprised in his/her mutation profile ranges from at least about 0.01% to at least about 10%. The ctDNA fraction of a sample disclosed herein is about at least 0.01%, 0.02%, 0.03%, 0.04%, 0.05%, 0.06%, 0.07%, 0.08%, 0.09%, 0.1%, 0.15%, 0.2%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, or any percentage in between.

In some embodiments, the generated mutation profile of a subject does not include mutation variants derived from cell-free nucleic acid molecules derived from a biological sample. In some embodiments, the mutation profile comprises genetic polymorphisms, such as missense variant, a nonsense variant, a deletion variant, an insertion variant, a duplication variant, an inversion variant, a frameshift variant, or a repeat expansion variant. In some embodiments, the mutation profile may comprise mutation variant derived from a fraction of cell-free nucleic acid molecules of a specific size range.

### Fragment Length Profile

In some embodiment, the length of ctDNA fragments is shorter than cell-free nucleic acid molecules derived from a healthy subject. In some embodiments, the length of ctDNA comprising at least one mutation is shorter than the length of cell free nucleic acid molecule containing a corresponding reference allele.

In some embodiments, the sequencing does not utilize bisulfite sequencing because it causes degradation of ctDNA fragments and prevents the preservation of the length distribution of ctDNAs. In some embodiments, the fragment length of a plurality of nucleic acids of the present disclosure (e.g., comprising a mixture cfDNA molecules derived from tumor or cancer tissue and healthy tissue, comprising cfDNA molecules only from healthy tissue, and/or comprising only ctDNA) can be from 1 to about 800 basepairs (bp), from about 50 bp to about 800 bp, from about 100 bp to about 200 bp, from about 120 bp to about 150 bp, from about 60 to about 500 bp, from about 80 to about 300 bp, from 90 to about 250 bp, from 80 to 170 bp, or from about 100 to about 150 bp. In some embodiments, the fragment length of a plurality of nucleic acids of the present disclosure (e.g., comprising a mixture cfDNA molecules derived from tumor or cancer tissue and healthy tissue, comprising cfDNA molecules only from healthy tissue, and/or comprising only ctDNA) can be at least 800 basepairs (bp), at least 700 basepairs, at least 600 basepairs, at least 500 basepairs, at least 400 basepairs, at least 300 basepairs, at least 200 basepairs, at least 150 basepairs, at least 100 basepairs, or at least 50 basepairs. In some embodiments, the fragment length of a plurality of nucleic acids of the present disclosure (e.g., comprising a mixture cfDNA molecules derived from tumor or cancer tissue and healthy tissue, comprising cfDNA molecules only from healthy tissue, and/or comprising only ctDNA) can be at most 800 basepairs (bp), at most 700 basepairs, at most 600 basepairs, at most 500 basepairs, at most 400 basepairs, at most 300 basepairs, at most 200 basepairs, at most 150 basepairs, at most 100 basepairs, or at most 50 basepairs. In some embodiments, the present disclosure provides an enrichment of the cell free nucleic acid samples based on selecting cell free molecules of a certain size. In some embodiments, the multimodal analysis comprises utilizing the mutation profile described herein and the fragment length profile by selectively including a plurality of nucleic acid molecules in the mutation profile based on their fragment length. In some embodiments, the multimodal analysis comprises utilizing the methylation profile described herein and the fragment length profile by selectively including a plurality of nucleic acid molecules in the methylation profile based on their fragment length. In some embodiments, the multimodal analysis comprises utilizing the mutation profile, methylation profile, and the fragment length profile together by selectively including a plurality of nucleic acid molecules in the mutation profile based on their fragment length and by selectively including a plurality of nucleic acid molecules in the methylation profile based on their fragment length respectively.

### Tumor Detection and Prognosis

The present disclosure provides methods and systems for determining whether a subject has or is at risk of having a disease, wherein the methods and systems comprise subjecting a plurality of nucleic acid molecules derived from a cell-free nucleic acid sample obtained from said subject to sequencing to generate at least one profile of (i) a methylation profile, (ii) a mutation profile, and (iii) a fragment length profile; and processing said at least one profile to determine whether said subject has or is at risk of said disease at a sensitivity of at least 80% or at a specificity of at least about 90%, wherein said cell-free nucleic acid sample comprises less than 30 ng/ml of said plurality of nucleic acid molecules. In some embodiments, the sensitivity is at least about 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, 99.7%, 99.8%, 99.9%, or any percentage in between the numbers. In some embodiments, the specificity is at least about 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or any percentage in between the numbers.

In some embodiments, the methods and systems can comprise subjecting a plurality of nucleic acid molecules derived from a cell-free nucleic acid sample obtained from said subject to sequencing to generate at least two profiles of (i) a methylation profile, (ii) a mutation profile, and (iii) a fragment length profile. The methods provide a sensitivity of at least about 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, 99.7%, 99.8%, 99.9%, or any percentage in between the numbers. In some embodiments, the sensitivity when using two profiles is increased by at least about 0.5%, 1%, 2%, 3%, 4%, 5%, 6%, 7%, 8%, 9%, 10%, or percentage in between any of the numbers compared to the sensitivity when using one profile. In some embodiments, the sensitivity when using three profiles is increased by at least about 0.5%, 1%, 2%, 3%, 4%, 5%, 6%, 7%, 8%, 9%, 10%, or percentage in between any of the numbers compared to the sensitivity when using two profiles.

Further, the methods provide a specificity of at least about 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or any percentage in between the numbers. In some embodiments, the specificity when using two profiles is increased by at least about 0.5%, 1%, 2%, 3%, 4%, 5%, 6%, 7%, 8%, 9%, 10%, or percentage in between any of the numbers compared to the specificity when using one profile. In some embodiments, the specificity when using three profiles is increased by at least about 0.5%, 1%, 2%, 3%, 4%, 5%, 6%, 7%, 8%, 9%, 10%, or percentage in between any of the numbers compared to the specificity when using two profiles.

The present disclosure provides methods and systems for processing a cell-free nucleic acid sample of a subject to determine whether said subject has or is at risk of having a disease, the methods and systems comprise providing said cell-free nucleic acid sample comprising a plurality of nucleic acid molecules; subjecting said plurality of nucleic acid molecules or derivatives thereof to sequencing to generate a plurality of sequencing reads; computer processing said plurality of sequencing reads to identify, for said plurality of nucleic acid molecules, (i) a methylation profile, (ii) a mutation profile, and (iii) a fragment length profile; and using at least said methylation profile, said mutation profile and said fragment length profile to determine whether said subject has or is at risk of having said disease. In some embodiments, the methods provide a sensitivity of at least about 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, 99.7%, 99.8%, 99.9%, or any percentage in between the numbers. The methods provide a specificity of at least about 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or any percentage in between the numbers.

The present disclosure provides methods and systems for determining a tissue origin of a tumor, comprising identifying a nucleotide sequence specific for a particular cancer (e.g., breast cancer, colon cancer, prostate cancer, HSNCC, or lung cancer) from which a fraction of cell-free nucleic acid molecules. In some embodiments, the fraction of the cell-free nucleic acid molecules is derived from ctDNA. In some embodiments, the methods provide a sensitivity of at least about 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, 99.7%, 99.8%, 99.9%, or any percentage in between the numbers. The methods provide a specificity of at least about 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or any percentage in between the numbers.

The present disclosure describes methods and systems for providing a prognosis to a subject after receiving a treatment for a disease and/or condition. For example, the treatment comprises a surgical removal of a tumor, a chemotherapy designed for a specific type of cancer, a radio therapy, or an immune therapy (e.g., TCR, CAR, etc.). In some embodiments, the methods or systems comprise subjecting a plurality of nucleic acid molecules derived from a cell-free nucleic acid sample obtained from said subject to sequencing to generate at least one profile of (i) a methylation profile, (ii) a mutation profile, and (iii) a fragment length profile; and monitoring or detecting minimal residual disease (MRD) based at least based on the at least one profile.

Once a subject is accurately diagnosed and receives a treatment to treat the cancer, such as surgical removal, chemotherapy, radio therapy, etc., it can be important to monitor the effectiveness of the treatment and predict the patient's survival rate. Further, it can be important to detect minimal residual disease of cancer cells.

In some embodiments, the method further comprises the step of adding a second amount of control DNA to the sample for confirming the immunoprecipitation reaction.

As used herein, the “control” may comprise both positive and negative control, or at least a positive control.

In some embodiments, the method further comprises the step of adding a second amount of control DNA to the sample for confirming the capture of cell-free methylated DNA.

In some embodiments, identifying the presence of DNA from cancer cells further includes identifying the cancer cell tissue of origin.

In some instances, tumor tissue sampling may be challenging or carry significant risks, in which case diagnosing and/or subtyping the cancer without the need for tumor tissue sampling may be desired. For example, lung tumor tissue sampling may require invasive procedures such as mediastinoscopy, thoracotomy, or percutaneous needle biopsy; these procedures may result in a need for hospitalization, chest tube, mechanical ventilation, antibiotics, or other medical interventions. Some individuals may not undergo the invasive procedures needed for tumor tissue sampling either because of medical comorbidities or due to preference. In some instances, the actual procedure for tumor tissue procurement may depend on the suspected cancer subtype. In other instances, cancer subtype may evolve over time within the same individual; serial assessment with invasive tumor tissue sampling procedures is often impractical and not well tolerated by patients. Thus, non-invasive cancer subtyping via blood test may have many advantageous applications in the practice of clinical oncology.

Accordingly, in some embodiments, identifying the cancer cell tissue of origin further includes identifying a cancer subtype. In some cases, the cancer subtype differentiates the cancer based on stage (e.g., early stage lung cancer treated with surgery vs late stage lung cancer treated with chemotherapy), histology (e.g., small cell carcinoma vs adenocarcinoma vs squamous cell carcinoma in lung cancer), gene expression pattern or transcription factor activity (e.g., ER status in breast cancer), copy number aberrations (e.g., HER2 status in breast cancer), specific rearrangements (e.g., FLT3 in AML), specific gene point mutational status (e.g., IDH gene point mutations), and DNA methylation patterns (e.g., MGMT gene promoter methylation in brain cancer).

In some embodiments, comparisons can be carried out genome-wide. In other embodiments, the comparisons can be restricted from genome-wide to specific regulatory regions, such as, but not limited to, long interspersed nuclear elements (LINEs), short interspersed nuclear elements (SINEs), long terminal repeats (LTRs), FANTOM5 enhancers, CpG Islands, CpG shores, CpG Shelves, or any combination of the foregoing. In other embodiments, the comparisons can be restricted from genome-wide to the background defined by enrichment of hypermethylated regions.

In some embodiments, the methods herein are for use in the detection of the cancer.

In some embodiments, the methods herein are for use in monitoring therapy of the cancer.

### Data Analysis Systems and Methods

The methods and systems disclosed herein may comprise algorithms or uses thereof. The one or more algorithms may be used to classify one or more samples from one or more subjects. The one or more algorithms may be applied to data from one or more samples. The data may comprise biomarker expression data. In some embodiments, the methods or systems comprise subjecting a plurality of nucleic acid molecules derived from a cell-free nucleic acid sample obtained from said subject to sequencing to generate at least one profile of (i) a methylation profile, (ii) a mutation profile, and (iii) a fragment length profile; and monitoring or detecting minimal residual disease (MRD) based on at least one profile. The methods disclosed herein may comprise assigning a classification to one or more samples from one or more subjects. Assigning the classification to the sample may comprise applying an algorithm to the methylation profile, mutation profile, and fragment length profile. In some cases, at least one profile is inputted to a data analysis system comprising a trained algorithm for classifying the sample as obtained from a subject which has a disease or minor injuries.

A data analysis system may be a trained algorithm. The algorithm may comprise a linear classifier. In some instances, the linear classifier comprises one or more of linear discriminant analysis, Fisher's linear discriminant, Naïve Bayes classifier, Logistic regression, Perceptron, Support vector machine, or a combination thereof. The linear classifier may be a support vector machine (SVM) algorithm. The algorithm may comprise a two-way classifier. The two-way classifier may comprise one or more decision tree, random forest, Bayesian network, support vector machine, neural network, or logistic regression algorithms.

The algorithm may comprise one or more linear discriminant analysis (LDA), Basic perceptron, Elastic Net, logistic regression, (Kernel) Support Vector Machines (SVM), Diagonal Linear Discriminant Analysis (DLDA), Golub Classifier, Parzen-based, (kernel) Fisher Discriminant Classifier, k-nearest neighbor, Iterative RELIEF, Classification Tree, Maximum Likelihood Classifier, Random Forest, Nearest Centroid, Prediction Analysis of Microarrays (PAM), k-medians clustering, Fuzzy C-Means Clustering, Gaussian mixture models, graded response (GR), Gradient Boosting Method (GBM), Elastic-net logistic regression, logistic regression, or a combination thereof. The algorithm may comprise a Diagonal Linear Discriminant Analysis (DLDA) algorithm. The algorithm may comprise a Nearest Centroid algorithm. The algorithm may comprise a Random Forest algorithm. In some embodiments, for discrimination of preeclampsia and non-preeclampsia, the performance of logistic regression, random forest, and gradient boosting method (GBM) is superior to that of linear discriminant analysis (LDA), neural network, and support vector machine (SVM).

The present disclosure provides methods and systems for determining whether a subject has or is at risk of having a disease, wherein the methods and systems comprises subjecting a plurality of nucleic acid molecules derived from a cell-free nucleic acid sample obtained from said subject to sequencing to generate at least one profile of (i) a methylation profile, (ii) a mutation profile, and (iii) a fragment length profile; and processing said at least one profile to determine whether said subject has or is at risk of said disease at a sensitivity of at least 80% or at a specificity of at least about 90%, wherein said cell-free nucleic acid sample comprises less than 30 ng/ml of said plurality of nucleic acid molecules. In some embodiments, the sensitivity is at least about 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, 99.7%, 99.8%, 99.9%, or any percentage in between the numbers. In some embodiments, the specificity is at least about 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or any percentage in between the numbers.

In some embodiments, the methods and systems can comprise subjecting a plurality of nucleic acid molecules derived from a cell-free nucleic acid sample obtained from said subject to sequencing to generate at least two profiles of (i) a methylation profile, (ii) a mutation profile, and (iii) a fragment length profile. The methods provide a sensitivity of at least about 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, 99.7%, 99.8%, 99.9%, or any percentage in between the numbers. In some embodiments, the sensitivity when using two profiles is increased by at least about 0.5%, 1%, 2%, 3%, 4%, 5%, 6%, 7%, 8%, 9%, 10%, or percentage in between any of the numbers compared to the sensitivity when using one profile. In some embodiments, the sensitivity when using three profiles is increased by at least about 0.5%, 1%, 2%, 3%, 4%, 5%, 6%, 7%, 8%, 9%, 10%, or percentage in between any of the numbers compared to the sensitivity when using two profiles.

Further, the methods provide a specificity of at least about 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or any percentage in between the numbers. In some embodiments, the specificity when using two profiles is increased by at least about 0.5%, 1%, 2%, 3%, 4%, 5%, 6%, 7%, 8%, 9%, 10%, or percentage in between any of the numbers compared to the specificity when using one profile. In some embodiments, the specificity when using three profiles is increased by at least about 0.5%, 1%, 2%, 3%, 4%, 5%, 6%, 7%, 8%, 9%, 10%, or percentage in between any of the numbers compared to the specificity when using two profiles.

The present disclosure provides methods and systems for processing a cell-free nucleic acid sample of a subject to determine whether said subject has or is at risk of having a disease, the methods and systems comprise providing said cell-free nucleic acid sample comprising a plurality of nucleic acid molecules; subjecting said plurality of nucleic acid molecules or derivatives thereof to sequencing to generate a plurality of sequencing reads; computer processing said plurality of sequencing reads to identify, for said plurality of nucleic acid molecules, (i) a methylation profile, (ii) a mutation profile, and (iii) a fragment length profile; and using at least said methylation profile, said mutation profile and said fragment length profile to determine whether said subject has or is at risk of having said disease. In some embodiments, the methods provide a sensitivity of at least about 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, 99.7%, 99.8%, 99.9%, or any percentage in between the numbers. The methods provide a specificity of at least about 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or any percentage in between the numbers.

The present disclosure describes methods and systems for providing a prognosis to a subject after receiving a treatment for a disease and/or condition. For example, the treatment comprises a surgical removal of a tumor, a chemotherapy designed for a specific type of cancer, a radio therapy, or an immune therapy (e.g., TCR, CAR, etc.). In some embodiments, the methods or systems comprise subjecting a plurality of nucleic acid molecules derived from a cell-free nucleic acid sample obtained from said subject to sequencing to generate at least one profile of (i) a methylation profile, (ii) a mutation profile, and (iii) a fragment length profile; and monitoring or detecting minimal residual disease (MRD) based on the at least one profile.

### Computer Systems

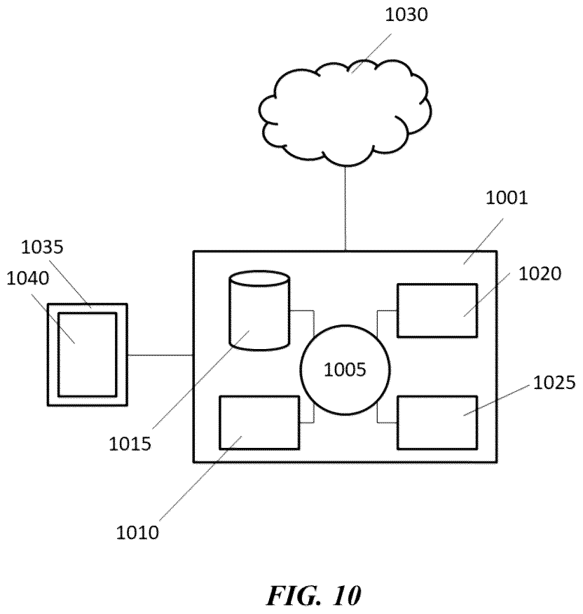

The present disclosure provides computer systems that are programmed to implement methods of the disclosure. FIG. 10 shows a computer system 1001 that is programmed or otherwise configured to identify, analyze, and compare sequences of hypermethylated cell-free nucleic acids (e.g., cfDNA). The computer system 1001 can be an electronic device of a user or a computer system that is remotely located with respect to the electronic device. The electronic device can be a mobile electronic device.

The computer system 1001 includes a central processing unit (CPU, also “processor” and “computer processor” herein) 1005, which can be a single core or multi core processor, or a plurality of processors for parallel processing. The computer system 1001 also includes memory or memory location 1010 (e.g., random-access memory, read-only memory, flash memory), electronic storage unit 1015 (e.g., hard disk), communication interface 1020 (e.g., network adapter) for communicating with one or more other systems, and peripheral devices 1025, such as cache, other memory, data storage and/or electronic display adapters. The memory 1010, storage unit 1015, interface 1020 and peripheral devices 1025 are in communication with the CPU 1005 through a communication bus (solid lines), such as a motherboard. The storage unit 1015 can be a data storage unit (or data repository) for storing data. The computer system 1001 can be operatively coupled to a computer network (“network”) 1030 with the aid of the communication interface 1020. The network 1030 can be the Internet, an internet and/or extranet, or an intranet and/or extranet that is in communication with the Internet. The network 1030 in some cases is a telecommunication and/or data network. The network 1030 can include one or more computer servers, which can enable distributed computing, such as cloud computing. The network 1030, in some cases with the aid of the computer system 1001, can implement a peer-to-peer network, which may enable devices coupled to the computer system 1001 to behave as a client or a server.

The CPU 1005 can execute a sequence of machine-readable instructions, which can be embodied in a program or software. The instructions may be stored in a memory location, such as the memory 1010. The instructions can be directed to the CPU 1005, which can subsequently program or otherwise configure the CPU 1005 to implement methods of the present disclosure. Examples of operations performed by the CPU 1005 can include fetch, decode, execute, and writeback.

The CPU 1005 can be part of a circuit, such as an integrated circuit. One or more other components of the system 1001 can be included in the circuit. In some cases, the circuit is an application specific integrated circuit (ASIC).

The storage unit 1015 can store files, such as drivers, libraries, and saved programs. The storage unit 1015 can store user data, e.g., user preferences and user programs. The computer system 1001 in some cases can include one or more additional data storage units that are external to the computer system 1001, such as located on a remote server that is in communication with the computer system 1001 through an intranet or the Internet.

The computer system 1001 can communicate with one or more remote computer systems through the network 1030. For instance, the computer system 1001 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 1001 via the network 1030.

Methods as described herein can be implemented by way of machine (e.g., computer processor) executable code stored on an electronic storage location of the computer system 1001, such as, for example, on the memory 1010 or electronic storage unit 1015. The machine executable or machine-readable code can be provided in the form of software. During use, the code can be executed by the processor 1005. In some cases, the code can be retrieved from the storage unit 1015 and stored on the memory 1010 for ready access by the processor 1005. In some situations, the electronic storage unit 1015 can be precluded, and machine-executable instructions are stored on memory 1010.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 1001, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1001 can include or be in communication with an electronic display 1035 that comprises a user interface (UI) 1040. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1005. The algorithm can, for example, identify, sequence, and analyze hypermethylated DNA regions.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

Alternative Embodiments

The disclosure is further understood through review of the numbered embodiments recited herein. Various embodiments contemplated herein may include, but need not be limited to, one or more of the following, and combinations thereof:

1. A method for cell-free nucleic acid processing, comprising: a. providing a cell-free nucleic acid molecule of a subject; b. subjecting said cell-free nucleic acid molecule or derivative thereof to conditions sufficient to increase a methylation level of said cell-free nucleic acid molecule or derivative thereof, thereby yielding a hypermethylated cell-free nucleic acid molecule; and c. identifying a sequence of said hypermethylated cell-free nucleic acid molecule or derivative thereof.

2. The method of claim 1, wherein said cell-free nucleic acid molecule in (a) is obtained from a blood of said subject.

3. The method in claim 2, wherein said blood comprises plasma, and wherein said cell-free nucleic acid molecule in (a) is obtained from said plasma.

4. The method of claim 1, wherein said identifying of (c) comprises sequencing said hypermethylated cell-free nucleic acid molecule.

5. The method of claim 4, wherein said sequencing comprises detecting methylation patterns.

6. The method of claim 5, wherein detecting methylation patterns comprises identifying highly methylated regions.

7. The method of any one of claims 1-6, further comprising immunoprecipitating said hypermethylated cell-free nucleic acid molecule before sequencing.

8. The method of claim 7, wherein said sequencing comprises sequencing said immunoprecipitated hypermethylated cell-free nucleic acid molecule.

9. The method of claim 7, wherein immunoprecipitating comprises using a methylated nucleic acid binder to bind said hypermethylated cell-free nucleic acid molecule.

10. The method of claim 9, wherein said methylated nucleic acid binder comprises an antibody for methylated nucleic acid.

11. The method of claim 10, wherein said antibody for methylated nucleic acid comprises an anti-5-Methylcytosine (5-mC) antibody.

12. The method of any one of claims 1-11, wherein said sequencing comprises subjecting said cell-free nucleic acid molecule or derivative thereof to a cfMeDIP-seq protocol.

13. The method of any one of claims 1-12, wherein said cell-free nucleic acid molecule or derivative thereof is subjected to in vitro conditions sufficient to increase a methylation level of said cell-free nucleic acid molecule.

14. The method of any one of claims 1-13, wherein said conditions sufficient to increase a methylation level of said cell-free nucleic acid molecule comprises an enzyme.

15. The method of claim 14, wherein the enzyme comprises CpG Methyltransferase.

16. The method of any one of claims 1-15, wherein said cell-free nucleic acid molecule in (a) is obtained from a body fluid comprising at least one of urine, amniotic fluid, cerebrospinal fluid, prostatic fluid, tear, or saliva.

17. The method of any one of claims 1-16, further comprising: a. providing a sheared genomic nucleic acid molecule, wherein said sheared genomic nucleic acid molecule is separate from said cell-free nucleic acid molecule; b. subjecting said sheared genomic nucleic acid molecule or derivative thereof to conditions sufficient to increase a methylation level of said sheared genomic nucleic acid molecule or derivative thereof, thereby yielding a hypermethylated genomic nucleic acid molecule; and c. identifying a sequence of said hypermethylated genomic nucleic acid molecule or derivative thereof.

18. The method of claim 17, further comprising, prior to (a), obtaining said sheared genomic nucleic acid molecule from said subject.

19. The method of claim 17, wherein said sheared genomic nucleic acid sample is not from said subject.

20. The method of claim 17, wherein said sheared genomic nucleic acid molecule comprises similar sequence length to that of cell-free nucleic acid.

21. The method of claim 17, wherein said identifying in c) comprises sequencing said hypermethylated genomic nucleic acid molecule or derivative thereof.

22. The method of claim 21, further comprising using said sequencing to identify a methylation pattern of said hypermethylated genomic nucleic acid molecule or derivative thereof.

23. The method of claim 22, further comprising using said sequencing to identify a highly methylated region of said hypermethylated genomic nucleic acid molecule, wherein said highly methylated region is a region that is at least 50% methylated.

24. The method of claim 23, further comprising processing said highly methylated regions identified in said hypermethylated cell-free nucleic acid with said highly methylated regions identified in said hypermethylated genomic nucleic acid and identifying a window of overlapping highly methylated regions.

25. The method of claim 24, wherein said window of overlapping highly methylated regions contains overlapping highly methylated sequences that are about 100 base pairs (bp) to 500 bp in length.

26. The method of claim 25, wherein said window of overlapping highly methylated regions contains overlapping highly methylated sequences that are about 250 bp to 350 bp in length.

27. The method of any one of claims 24-26, wherein a plurality of identified windows of overlapping highly methylated regions collectively comprises from about 10% to about 20% of a whole genome.

28. The method of any one of claims 24-26, wherein said window comprises from about 0.5 million to 2.5 million highly methylated regions.

29. A method of identifying differentially methylated regions (DMRs), comprising analyzing and comparing methylation patterns of a plurality of windows of overlapping sequence reads between a diseased sample and a control sample, and identifying hyper-or hypomethylated regions in said diseased sample as differentially methylated regions (DMRs), as compared to said control sample.

30. The method of claim 29, wherein analyzing methylation patterns of said plurality of windows of overlapping sequence reads comprises immunoprecipitating cell-free nucleic acid in said diseased sample and then analyzing methylation patterns of said immunoprecipitated cell-free nucleic acid.

31. The method of claim 29, wherein said method identifies DMRs that are not found using a whole-genome as a background.

32. The method of claim 29, wherein said method yields a larger number of DMRs as compared to using a whole-genome as a background.

33. The method of claim 29, wherein said method yields at least twice said number of DMRs as compared to using a whole-genome as a background.

34. The method of claim 29, wherein said method can identify DMRs more quickly than using a whole-genome as a background.

35. A method of analyzing a sample, comprising analyzing said differentially methylated regions (DMRs) identified in any one of claims 29-34 in said diseased sample, wherein said method identifies said sample as a diseased sample or a normal sample.

36. The method of claim 35, wherein said method provides better distinction compared to using genome-wide DMRs.

37. The method of claim 35, wherein said method provides a distinction between a cancerous sample and a normal sample.

38. The method of claim 37, wherein said method provides a distinction between different types of cancer.

39. The method of claim 1, further comprising providing a therapeutic intervention to said subject upon identifying said sequence.

40. The method of claim 39, wherein providing said therapeutic intervention comprises providing a therapy to said subject.

41. The method of claim 39, wherein providing said therapeutic intervention comprises providing a report that identifies said sequence of said hypermethylated cell-free nucleic acid molecule or derivative thereof.

42. The method of claim 39, wherein providing said therapeutic intervention comprises providing a report that provides a therapeutic recommendation to said subject for treating a disease associated with said hypermethylated cell-free nucleic acid molecule or derivative thereof.

43. The method of claim 39, wherein providing said therapeutic intervention comprises treating said subject for a disease associated with said hypermethylated cell-free nucleic acid molecule or derivative thereof.

44. The method of claim 42 or 43, wherein said disease is cancer.

### EXAMPLES

### Example 1: Provision of Cell-Free DNA

This example shows methods and systems for identifying all the genomic regions that are the most prone to be pulled down by a methylated DNA binder.

A 5mC antibody was used as a methylated DNA binder to identify a set of background genomic regions that, when methylated, were enriched by the binder. In this example, the 5mC antibody was used for the cfMeDIP-seq assay to distinguish cancer and control samples using plasma cell-free DNA (cfDNA). Using this validated set of genomic regions instead of the whole genome as a background significantly improved the ability to identify differentially methylated regions (DMRs) between conditions, such as cancer patients versus healthy donors or between different cancer types.

Using commercially available normal donor plasma (Cedarlane, Cat # IPLASNAE50ML) as the starting material, cfDNA extraction was carried out using the Apostle MiniMax High Efficiency cfDNA Isolation Kit (Apostle, Cat #C40605) following manufacturer's protocol. Eluted cfDNA, to be known as native cfDNA, was quantified via Qubit prior to proceeding to the in vitro methylation step.

Commercially available K562 genomic DNA (Promega, Cat #DD2011) was sheared on a Covaris LE-220 ultrasonicator before it was size-selected via AMPure XP beads (Beckman Coulter, Cat #A63882) using 1.2X-1.7X bead ratio to generate DNA that mimicked sizing of cfDNA. The size-selected, sheared DNA, to be known as cfDNA mimic, was quantified via Qubit prior to proceeding to the in vitro methylation step.

As shown in FIG. 14, samples were used from both native cfDNA and from sheared genomic DNA (cfDNA mimic). The native cfDNA allowed a closer representation of the fragmentation patterns observed in clinical samples, while the cfDNA mimic allowed a better genome-wide representation. Both native cfDNA and cfDNA mimics were fully methylated in vitro and subjected to the cfMeDIP-seq protocol in order to identify all genomic regions able to be pulled down by the 5mC antibody.

Methylation reactions using 150 ng of native cfDNA and 150 ng of cfDNA mimic as the starting material were set up using CpG Methyltransferase (M.SssI) (ThermoFisher Scientific, Cat #EM0821), following the manufacturer's protocol. Due to the low amount of cfDNA available, a surrogate control sample was also set up alongside the native cfDNA and cfDNA mimic to test for proper methylation. This surrogate control sample, an amplicon generated in-house, is available in larger quantities and has a restriction site that corresponds to methylation-sensitive restriction enzyme HpyCH4IV. For the in vitro methylation, the volume of the starting material was supplemented to 16.6 μL with nuclease-free water before it was mixed with the following mastermix: 2 μL of 10X M.SssI Buffer, 0.4 μL 50X SAM and 1 μL of M.SssI Enzyme. The reaction was incubated at 37° C. for 15 min, followed by inactivation at 65° C. for 20 min. The methylated DNA was purified using Qiagen MinElute PCR Clean up kit (Qiagen, Cat #28004) following manufacturer's protocol before being quantified via Qubit.

The methylated surrogate control sample and an aliquot of the original surrogate control sample were subjected to methylation sensitive restriction digest using restriction enzyme HpyCH4IV (NEB, Cat #R0619S) following manufacturer's protocol. After purification of the digested product using the Qiagen MinElute PCR Clean up kit, through TapeStation profile, it was verified that there was digestion of the original surrogate sample (multiple smaller products) but no digestion of the methylated surrogate control (single larger product) indicating successful in vitro methylation.

Commercially purchased, biobanked plasma samples corresponding to cancer and normal control patients, ranging in volume from 1-3 mL were extracted using the Apostle MiniMax High Efficiency cfDNA Isolation Kit (Apostle, Cat #C40605) following manufacturer's protocol. The isolated DNA was quantified via Qubit and profiled using Agilent TapeStation cfDNA assay kit to ensure % cfDNA, as determined by the TapeStation Analysis software, was ≥50%.

Both the immunoprecipitated (IP'ed) fraction and the input control were sequenced. Using 10 ng of methylated native cfDNA and methylated cfDNA mimic, each carried out in triplicate and supplemented with 0.1 ng of spike-in control DNA, library preparation was carried out using the Kapa Hyper Prep Kit in combination with the IDT xGen CS Adapter (IDT, Cat #1080799), following manufacturer's protocol with minor modifications. In brief, after end-repair and A-tailing, 0.327 μM of xGen CS adapter was ligated to the DNA following an incubation of 30 mins at 20° C. After purification of the adapter ligated DNA using AMPure XP beads, 5% of the DNA was saved as the input control. The remaining DNA was combined with 2 filler DNA to increase starting DNA amounts to 100 ng prior to MeDIP. MeDIP was carried out as previously published (Shen, S. Y., Burgener, J. M., Bratman, S. V., & De Carvalho, D. D. (2019). Preparation of cfMeDIP-seq libraries for methylome profiling of plasma cell-free DNA. Nature protocols, 14 (10), 2749-2780), with PCR cycles for the final IP'ed library amplification set to 15 cycles using IDT xGen UDI primers (IDT, Cat #10005922) prior to size selection with AMPure XP beads. The previously saved input control DNA was also amplified using the same PCR mastermix and protocol used for MeDIP, reducing the PCR cycle number to 10 cycles.

For the cfDNA extracted from cancer and control patients, an aliquot of 10 ng of cfDNA from each patient was supplemented with 0.1 ng of spike-in control DNA and used for library preparation and MeDIP as detailed above with minor modifications. The IP'ed libraries were amplified for 14 cycles before cleaned up with AMPure XP beads. The saved input control for these samples was not sequenced, just stored at −20° C. The final libraries from cfMeDIP-seq were sequenced on the NovaSeq 6000 with configuration of paired-end 100 bp.

To analyze the data, firstly, for the six fully in vitro methylated samples, the IP'ed and Input Control libraries were put through our pipeline which performs standard bioinformatics steps including trimming of raw reads in FASTQ files, aligning them to the human genome build hg38 to generate BAM files, and generation of (normalized) aligned read counts which measured the methylation signal in genomic windows of size 300-bp.

Using a standard peak calling algorithm, all the genome-wide regions enriched in the IP'ed fraction were identified and compared to the input control. These regions were defined as the “novel background” file, also known as the background region. The high methylation signal peaks/genomic regions from the fully in vitro methylated samples were identified with the standard MACS2 peak-calling software using the “narrow peaks” settings with q-value<0.01. Other thresholds, such as q<0.1, can also be used. The three IP'd libraries originating from native cfDNA were compared to the corresponding Input Controls, and the same was done for libraries from the three cfDNA mimic samples. The peaks from these two sets of samples were merged and the genomic windows of size 300-bp overlapping these narrow peaks were identified. These windows (n=1,218,413) constitute the background region, that was experimentally validated to be efficiently pulled down by the 5mC antibody. This background region was significantly smaller than the whole genome (n=9,583,339 windows).

Then, to test the utility of the background region file, the methylation signal from these 300-bp windows was obtained from patient samples with breast cancer (n=14), colorectal cancer (n=15) or lung cancer (n=30), as well as from control samples (n=59), with the MEDIPS software in both Counts and RPKM (Reads Per Kilobase per Million mapped reads) format. Separately, Counts and RPKMs were also obtained with these same 118 samples for all 300-bp windows from chromosomes 1 to 22 to obtain the “genome-wide” methylation signal.

Next, all the cancer samples (n=59) were compared to all the control samples (n=59) to get Differentially Methylated Regions (DMRs)/300-bp windows that either gain methylation in cancer, termed “Hyper DMRs” and those that lose methylation in cancer, termed “Hypo DMRs”. These DMRs were identified with the DESeq2 software using the 300-bp windows in background region, and separately with the 300-bp windows genome-wide, using the same threshold (q<0.1) with both backgrounds.

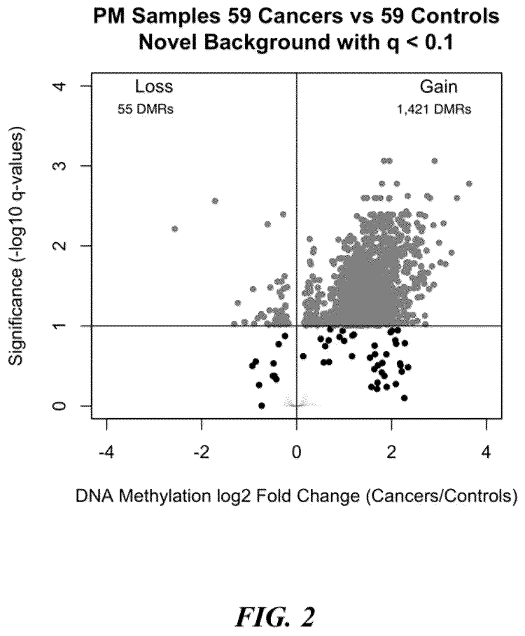

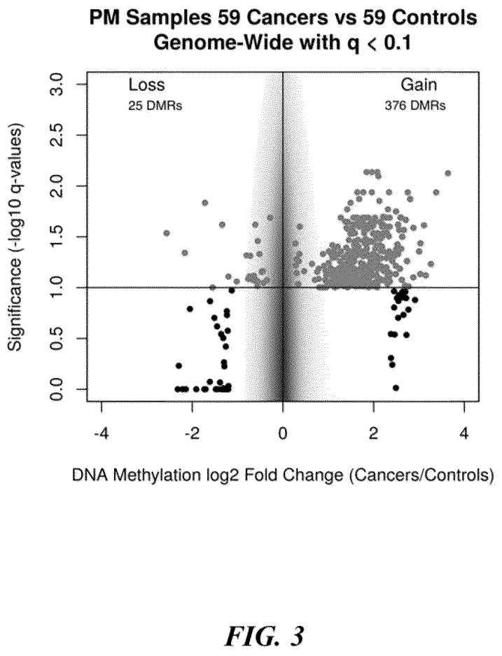

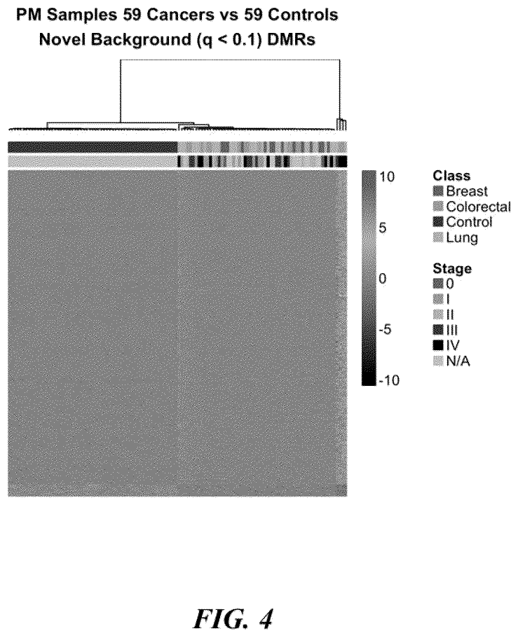

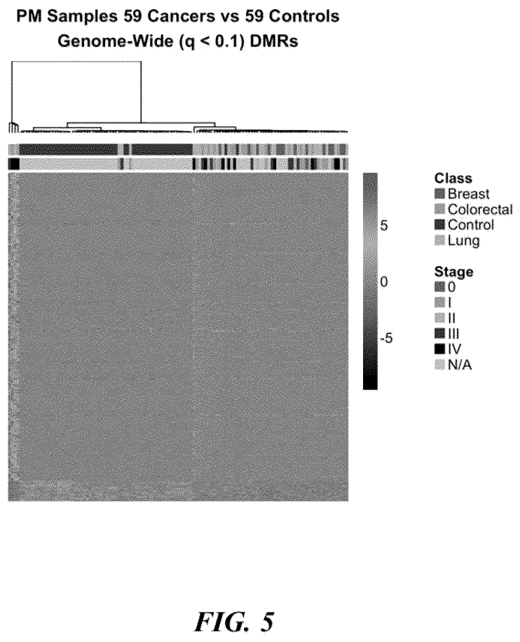

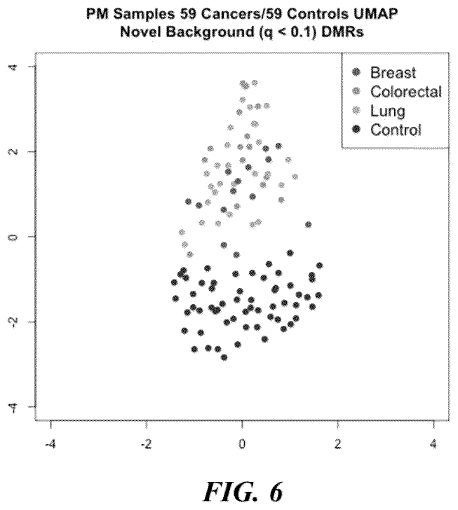

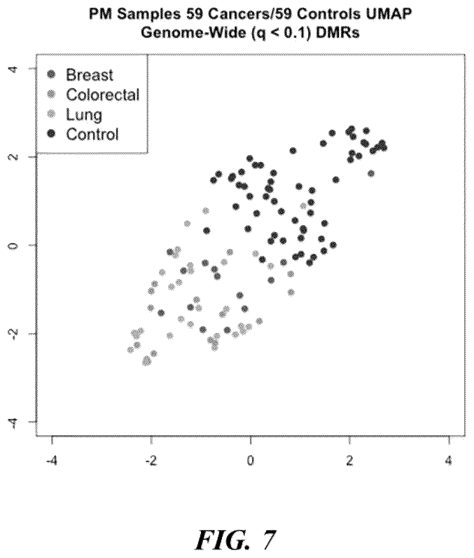

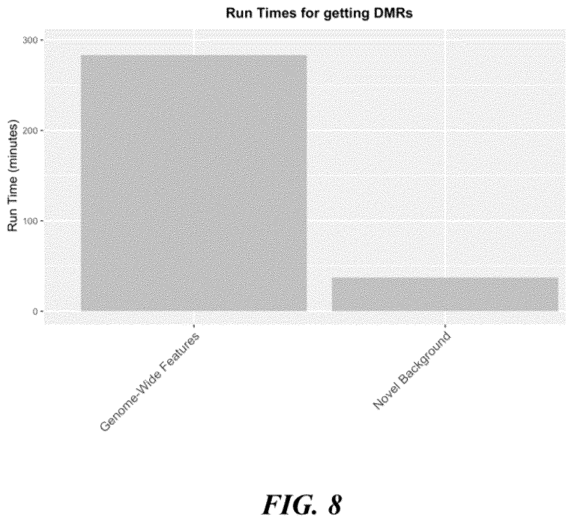

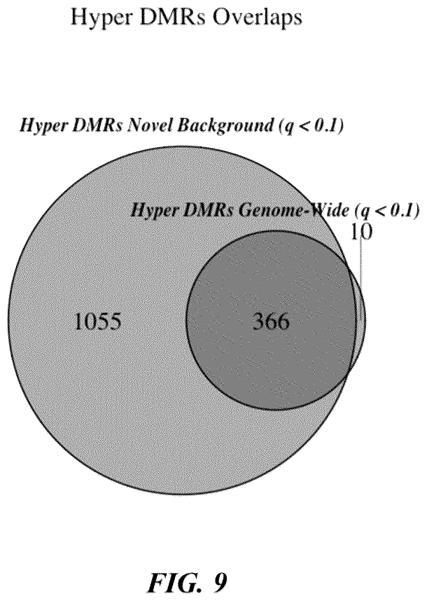

The background region file allowed for the identification of a much larger number of DMRs between cases and controls as compared to using the whole-genome as a background, as shown in the volcano plot in FIG. 2 and FIG. 3, highlighting the power of identifying DMRs between conditions as compared to using the whole genome as a background. As shown in the Venn diagram in FIG. 9, more DMRs were identified by using a background region as compared to using the whole-genome as a background, with majority of genome-wide DMRs overlapping with those identified with the background region. Moreover, the background region file allowed for computation of cancer versus control DMRs much faster (FIG. 8). Finally, the set of DMRs identified using the background region file was more efficient in distinguishing cases versus controls than the limited set of DMRs identified using the whole genome background. This was evident in the heatmaps showing the Z-scores of the RPKMs obtained from the MEDIPS software. As shown in FIG. 4, DMRs from the background region showed that various cancer samples can be clustered distinctly from the control samples, while with genome-wide DMRs (FIG. 5), some cancer cases clustered together with the healthy controls. The same standard clustering algorithm (Ward with Euclidean distance) was used for FIG. 4 and FIG. 5. Plots generated by the Uniform Manifold Approximation and Projection (UMAP), (FIG. 6, and FIG. 7) also showed that DMRs from the background region was more efficient in distinguishing the cancer and control samples as compared to genome-wide DMRs.

Taken together, this data showed that the DMRs generated from the background region identified from the fully methylated samples more clearly separated cancer and control samples than the DMRs generated genome-wide. In addition, the generation of these DMRs was much faster and less computationally intense. This experiment showed the ability to identify regions of the genome that work well with the methylation assay for the ultimate goal of accurately detecting tumor signal in patient samples.

In conclusion, there was a more efficient and accurate interpretation of experiments using a modified DNA binder, such as a 5mC antibody in cfMeDIP-seq experiments. This invention can improve any method that relies on affinity purification of modified DNA, such as cfMeDIP-seq, MDB-seq, 5hmC antibodies (hMeDIP-seq), 5hmC-SEAL, among others.

### Example 2: Peak Calling Methodology

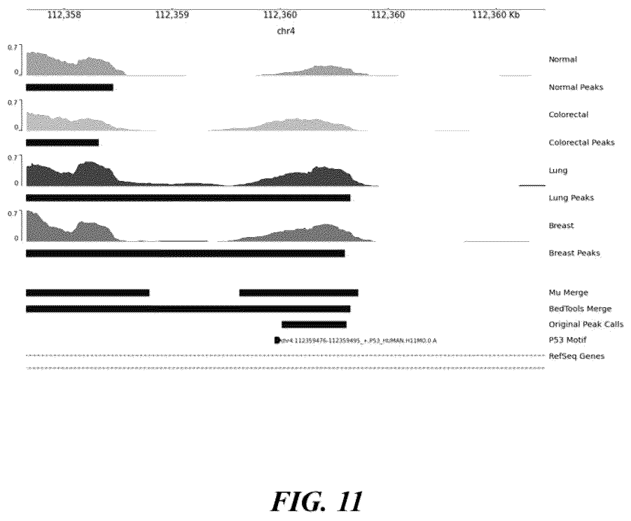

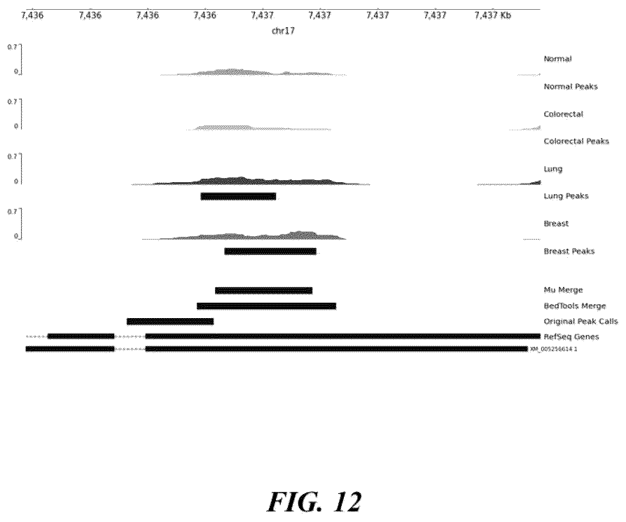

De nova peak calling was performed on a per sample basis using an open-source peak calling tool, MACS2. MACS2 “callpeak” function can be called with the “broad” or “narrow” option. In this example, MACS2 “callpeak” function was called with the “broad” option enabled, a “broad-cutoff”' of 0.1, a q-value threshold of 0.01, and with the option to keep duplicates that have not already been removed by the upstream pipeline. Input sample Binary Alignment Maps (BAMs) were read filtered for both Unique Molecular Identifiers (UMI) and optical duplication. Peak annotations were then produced by an algorithm that determines signal that differs significantly from the background mean coverage. The result was a peak annotation file that contains coordinates which represent a peak “region of interest” (ROI). The peak ROI represented a genomic location to observe methylation signal in a sample, as shown in FIG. 11 and FIG. 12. The “broad” option within MACS2 was able to generate new peak ROI that were not captured using the MACS2 callpeak with the “narrow” function.

De novo peak calls were obtained from three fully methylated samples. Then, an open-source tool, mumerge, which is a probabilistically-principled means of merging ROI across multiple samples, was used to combine per-sample peak ROI into one consensus set of feature annotations. Mumerge treated the ROI as probability distributions, taking into account any differing conditions (e.g., cell type, treatment, technical replicate) between samples, to produce a joint probability distribution that describes the highest likelihood position for that ROI. As shown in FIG. 11 and FIG. 12, this method resulted in higher resolution than simply merging all ROI that overlap to create the consensus annotation set, which has a propensity to inflate the size of the true ROI. This method increased resolution of the feature rather than being offset to the tail of the true peak ROI. Furthermore, the probabilistic feature was important to determine the relative proximity of changing ROI to other genomic features of interest, such as the P53 transcription factor motif in the annotation track shown in FIG. 11.

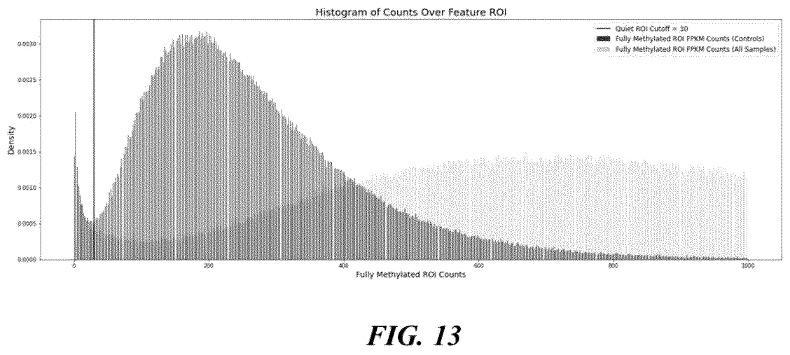

Once the consensus file has been generated, counts were generated over these peak ROI to use in feature selection and diagnostic algorithms, as shown in FIG. 13. Leveraging this approach, regions that are “quiet”, or have very few reads in control samples, can be derived, which can be used to determine change in response to a medical condition, such as cancer. Increased resolution over methylation ROI will better discriminate between healthy and disease states given the very low number of reads that will be captured coming from ctDNA.

Although preferred embodiments of the invention have been described herein, it will be understood by those skilled in the art that variations may be made thereto without departing from the spirit of the invention or the scope of the appended claims. All documents disclosed herein, including those in the following reference list, are incorporated by reference.

1-43. (canceled)

44. A method for generating a library of methylated regions, comprising:
a) subjecting a first population of nucleic acid molecules or derivatives thereof to conditions sufficient to increase methylation levels of at least a portion of said first population of nucleic acid molecules or derivatives thereof, thereby yielding a second population of nucleic acid molecules comprising hypermethylated nucleic acid molecules;
b) incubating at least a portion of said second population of nucleic acid molecules to enrich for methylated nucleic acid molecules of said second population of nucleic acid molecules; and
c) processing said methylated nucleic acid molecules of said second population of nucleic acid molecules to generate a library of methylated regions.

45. The method of claim 44, wherein said nucleic acid molecules or derivatives thereof are cell-free nucleic acid molecules.

46. The method of claim 44, wherein said nucleic acid molecules or derivatives thereof are genomic nucleic acid molecules.

47. The method of claim 44, wherein said first population of nucleic acid molecules or derivatives thereof comprises a first subpopulation of nucleic acid molecules or derivatives thereof and a second subpopulation of nucleic acid molecules or derivatives thereof.

48. The method of claim 47, wherein said first subpopulation of nucleic acid molecules or derivatives thereof comprises cell-free nucleic acid molecules and said second subpopulation of nucleic acid molecules or derivatives thereof comprises genomic nucleic acid molecules.

49. The method of claim 44, wherein said processing of (c) comprises subjecting said methylated nucleic acid molecules of said second population of nucleic acid molecules to conditions sufficient to amplify said methylated nucleic acid molecules of said second population of nucleic acid molecules, thereby generating amplified methylated nucleic acid molecules.

50. The method of claim 49, further comprising sequencing said amplified methylated nucleic acid molecules to generate a plurality of sequencing reads.

51. The method of claim 50, further comprising obtaining a portion of said second population of nucleic acid molecules, thereby generating control nucleic acid molecules.

52. The method of claim 51, further comprising subjecting said control nucleic acid molecules to amplification and sequencing under sufficient conditions to generate a plurality of control sequencing reads.

53. The method of claim 52, further comprising comparing said plurality of sequencing reads to said plurality of control sequencing reads to confirm enrichment of methylated nucleic acid molecules of said second population of nucleic acid molecules.

54. The method of claim 44, further comprising providing (i) sequencing reads from one or more cell-free nucleic acid samples of one or more subjects and (ii) sequencing reads of one or more cell-free nucleic acid samples of one or more healthy subjects.

55. The method of claim 54, wherein said sequencing reads of said one or more subjects are associated with enriched methylated nucleic acid molecules of said one or more subjects and said sequencing reads of said one or more healthy subjects are associated with enriched methylated nucleic acid molecules of said one or more healthy subjects.

56. The method of claim 54, further comprising mapping said sequencing reads of said one or more subjects to said library of methylated regions, thereby generating a first methylation pattern of methylated nucleic acid molecules associated with said one or more subjects.

57. The method of claim 56, further comprising mapping said sequencing reads of said one or more healthy subjects to said library of regions, thereby generating a second methylation pattern of methylated nucleic acid molecules associated with said one or more healthy subjects.

58. The method of claim 57, wherein said first methylation pattern and said second methylation pattern comprise unmethylated states or methylated states.

59. The method of claim 57, further comprising comparing said first methylation pattern to said second methylation pattern to generate one or more differentially methylated regions (DMRs).

60. The method of claim 59, wherein said one or more DMRs are used to classify a diseased sample and a healthy sample.

61. The method of claim 54, wherein said one or more subjects have or are suspected of having a disease or condition.

62. The method of claim 61, wherein said disease or said condition is a cancer or a tumor.

63. The method of claim 50, wherein said sequencing comprises narrow peak calling or broad peaking calling to identify methylation signals of said plurality of sequencing reads.
